SEQUENCE LISTING (14 sequences)

SEQ ID NO: 1 - DNA, 743 bp, Homo Sapiens
  misc_feature: TSLP
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo Sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo Sapiens
  MISC_FEATURE: LCDR1
GGNNLGSKSVH

SEQ ID NO: 4 - protein, 7 aa, Homo Sapiens
  MISC_FEATURE: LCDR2
DDSDRPS

SEQ ID NO: 5 - protein, 11 aa, Homo Sapiens
  MISC_FEATURE: LCDR3
QVWDSSSDHVV

SEQ ID NO: 6 - protein, 5 aa, Homo Sapiens
  MISC_FEATURE: HCDR1
TYGMH

SEQ ID NO: 7 - protein, 17 aa, Homo Sapiens
  MISC_FEATURE: HCDR2
VIWYDGSNKHYADSVKG

SEQ ID NO: 8 - protein, 13 aa, Homo Sapiens
  MISC_FEATURE: HCDR3
APQWELVHEAFDI

SEQ ID NO: 9 - DNA, 366 bp, Homo Sapiens
  misc_feature: Heavy Chain VH
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo Sapiens
  MISC_FEATURE: Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo Sapiens
  misc_feature: Light Chain VL
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo Sapiens
  MISC_FEATURE: Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 5 aa, Artificial Sequence
  Synthetic peptide
DYWMH

SEQ ID NO: 14 - protein, 19 aa, Artificial Sequence
  Synthetic peptide
HIKSKTDAGTTDYAAPVKG

(19) United States
(12) Patent Application Publication
(10) Pub. No.: US 2023/0081261 A1
(43) Pub. Date: Mar. 16, 2023

(54) FORMULATIONS OF HUMAN ANTI-TSLP ANTIBODIES AND METHODS OF USING THE SAME

(71) Applicant: AMGEN INC., Thousand Oaks, CA (US)

(72) Inventors: Lauren Roschen, Camarillo, CA (US); Jennifer Litowski, Arlington, MA (US)

(21) Appl. No.: 17/760,470

(22) PCT Filed: Feb. 18, 2021
(86) PCT No.: PCT/US2021/018561

§ 371 Date: Aug. 10, 2022

Related U.S. Application Data
(60) Provisional application No. 62/978,201, filed on Feb. 18, 2020.

(51) Int. Cl. C07K 16/24; A61K 47/12; A61K 47/18; A61K 47/26; A61K 9/00; A61K 9/08; A61P 29/00

(52) U.S. Cl. CPC C07K 16/246; A61K 47/12; A61K 47/183; A61K 47/26; A61K 9/0019; A61K 9/08; A61P 29/00; C07K 2317/565

(57) ABSTRACT

Provided herein are compositions comprising greater than about 100 mg/mL of an anti-TSLP antibody, a surfactant, proline, and a buffer comprising greater than about 100 mg/mL of an anti-TSLP antibody, a surfactant, proline, and a buffer. Methods for treating an inflammatory disease in a subject are further provided.

### CROSS REFERENCE TO RELATED APPLICATIONS

The present application is a national stage of PCT/US21/18561, filed Feb. 18, 2021 which claims the priority benefit of U.S. Provisional Patent Application No. 62/978,201, filed Feb. 18, 2020, herein incorporated by reference in its entirety.

### FIELD OF THE DISCLOSURE

The invention relates to human anti-TSLP monoclonal antibodies, including high-concentration aqueous formulations of tezepelumab and biosimilars thereof.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 9,906 byte ASCII (Text) file named “55238_Seqlisting.txt”; created on Aug. 8, 2022.

### BACKGROUND

### Brief Description of Related Technology

In a recent phase 2, randomized, double-blind, placebo-controlled clinical trial, tezepelumab (also known as AMG 157 and MED9929) was administered to humans at doses ranging from 70 mg to 280 mg. Subjects who received tezepelumab demonstrated lower rates of clinically significant asthma exacerbations than those who received placebo.

Increasing concentrations of protein in drug formulations can cause problems with stability, for example protein aggregation resulting in formation of high molecular weight species (HMWS). HMWS, particularly those that conserve most of the native configuration of the monomer counterpart, can be of particular concern in some protein formulations. Aggregation can also potentially affect the subcutaneous bioavailability and pharmacokinetics of a therapeutic protein.

Filling and finishing operations, as well as administration of drug product, can involve steps of flowing protein solutions through piston pumps, peristaltic pumps, or needles for injection. Such processes can impart shear and mechanical stresses, which can cause denaturation of proteins and result in aggregation. This phenomenon can be exacerbated as protein solutions become more concentrated.

### SUMMARY

Provided herein is an improved formulation for anti-TSLP antibodies having increased stability and low viscosity while containing a high concentration of antibody.

One aspect of the disclosure is a composition comprising greater than about 100 mg/mL of an anti-TSLP antibody, a surfactant, proline, and a buffer. In exemplary aspects, the anti-TSLP antibody is present in the composition at a concentration less than about 200 mg/mL or less than about 150 mg/mL. In exemplary aspects, the anti-TSLP antibody is present in the composition at a concentration of about 110 mg/mL to about 140 mg/mL. In exemplary aspects, the anti-TSLP antibody is present in the composition at a concentration about 110 mg/mL±10% or about 140 mg/mL±10%. Optionally, the anti-TSLP antibody is present in the composition at a concentration of about 105 mg/mL to about 115 mg/mL. In exemplary aspects, the surfactant is amphipathic and nonionic. In various aspects, the surfactant is a polysorbate, e.g., polysorbate 20 or polysorbate 80 or a mixture thereof. In exemplary instances, the surfactant is present in the composition at a concentration less than or about 0.015% (w/v)±0.005% (w/v), e.g., about 0.005% (w/v) to about 0.015% (w/v) surfactant. In some instances, the concentration of the surfactant is about 0.005% (w/v), 0.010% (w/v), or 0.015% (w/v). In exemplary aspects, the composition comprises less than about 3.0% (w/v) proline, e.g., about 2.4% (w/v) to about 2.8% (w/v) proline or about 2.5% (w/v) to about 2.8% (w/v) proline. In exemplary instances, the proline is L-proline. In certain aspects, proline is the only amino acid present in the composition. In exemplary aspects, the buffer is selected from the group consisting of: succinate, glutamate, histidine, and acetate. In preferred instances, the buffer is acetate. In exemplary aspects, the composition comprises about 1 mM to about 50 mM buffer, e.g., about 10 mM to about 30 mM buffer, optionally, about 15 mM to about 30 mM buffer, about 20 mM to about 30 mM buffer, or about 10 mM to about 25 mM buffer. Optionally, the buffer comprises about 20 mM to about 2 mM buffer (e.g., about 20 mM to about 28 mM buffer, about 23 mM to about 28 mM, about 24 mM to about 28 mM). In exemplary aspects, the composition comprises not more than 0.001% (w/v) of a sugar or citrate, optionally, wherein the sugar is a disaccharide, e.g., trehalose and sucrose. In exemplary aspects, the composition is a liquid, and, optionally, the pH is less than about 6.0, optionally, less than about 5.5. In certain aspects, the pH is about 4.5 to about 5.5 or about 4.8 to about 5.4 or about 4.9, about 5.2, or about 5.4. In exemplary aspects, the composition is characterized by a reduced viscosity, relative to liquid composition not comprising proline. For example, the composition, in some instances, is characterized by a viscosity of less than about 24 cP at about 20° C. to about 25° C. when the concentration of the anti-TSLP antibody is less than 155 mg/mL, optionally, ˜6 cP when the concentration of the anti-TSLP antibody is about 110 mg/mL or about 15 cP when the concentration of the anti-TSLP antibody is about 140 mg/mL. In some aspects, the composition is characterized by a viscosity of about 5 cP to about 20 cP. In various instances, the composition is isotonic or has an osmolality in a range of about 200 mOsm/kg to about 500 mOsm/kg, or about 225 mOsm/kg to about 400 mOsm/kg, or about 250 mOsm/kg to about 350 mOsm/kg. In exemplary instances, the composition is suitable for short term storage at 25° C., 30° C., or at 40° C., or long term storage at about -30° C. or about 2° C. to about 8° C. For example, less than 0.5% of the therapeutic protein is degraded after 6 months of storage at 2° C. to 8° C. as determined by Size Exclusion Chromatography (SEC), optionally, wherein the therapeutic protein is contained in glass vials or syringes. Also, for example, less than 5% of the antibody is degraded after about 24 months to about 36 months of storage at 2° C. to 8° C. as determined by Size Exclusion Chromatography (SEC), optionally, wherein less than 2% of the antibody is degraded after 24 months or 36 months of storage at 2° C. to 8° C. In various aspects, less than 5% of the antibody is degraded after at least 2 weeks (optionally, after at least 1 month, after at least 2 months, after at least 3 months, after at least 4 months, after at least 5 months or after at least 6 months) of storage at about 25° C., as determined by SEC. In various instances, less than 5% of the antibody is degraded after about 24 months to about 36 months of storage at 2° C. to 8° C. followed by at least 2 weeks or at least 1 month or at least 2 months of storage at about 25° C., as determined by SEC. In exemplary instances, the anti-TSLP antibody is an IgG2 antibody. In some aspects, the anti-TSLP antibody specifically binds to a TSLP polypeptide as set forth in amino acids 29-159 of SEQ ID NO: 2. Optionally, both binding sites of the antibody have identical binding to TSLP. In exemplary instances, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In exemplary aspects, the anti-TSLP antibody comprises: (A) a light chain variable domain selected from the group consisting of: (i) a sequence of amino acids at least 80% identical to SEQ ID NO:12; (ii) a sequence of amino acids encoded by a polynucleotide sequence that is at least 80% identical to SEQ ID NO:11; (iii) a sequence of amino acids encoded by a polynucleotide that hybridizes under moderately stringent conditions to the complement of a polynucleotide consisting of SEQ ID NO:11; and (B) a heavy chain variable domain selected from the group consisting of: (i) a sequence of amino acids that is at least 80% identical to SEQ ID NO:10; (ii) a sequence of amino acids encoded by a polynucleotide sequence that is at least 80% identical to SEQ ID NO:9; (iii) a sequence of amino acids encoded by a polynucleotide that hybridizes under moderately stringent conditions to the complement of a polynucleotide consisting of SEQ ID NO:9; or (C) a light chain variable domain of (A) and a heavy chain variable domain of (B).

Another aspect of the disclosure is a composition comprising about 110 mg/mL to about 140 mg/mL tezepelumab, about 0.01% (w/v)±0.005% (w/v) polysorbate 80, about 2.4% (w/v) to about 2.8% (w/v) L-proline, and about 20 mM to about 28 mM acetate, wherein the viscosity of the composition is less than about 20 cP and the pH is less than about 5.5. Optionally, the pH is 5.2, optionally, wherein the viscosity is about 15 cP at 20° C. to about 25° C.

Another aspect of the disclosure is a composition comprising about 110 mg/mL of an anti-TSLP antibody, 0.01% (w/v) polysorbate 80, about 2.4% (w/v) to about 2.8% (w/v) L-proline, and about 20 mM to about 28 mM acetate, wherein the composition has a pH of about 5.2. Optionally, the composition comprises about 22 mM to about 26 mM acetate or about 24 mM to about 26 mM.

Another aspect of the disclosure is a composition comprising about 140 mg/mL of an anti-TSLP antibody, 0.01% (w/v) polysorbate 80, about 2.5% (w/v) to about 2.8% (w/v) L-proline, and about 20 mM to about 28 mM acetate, wherein the composition has a pH of about 5.2. Optionally, the composition comprises about 25 mM to about 26 mM acetate.

In various embodiments, the composition comprises 110 mg/mL anti-TSLP antibody, 24 mM acetate, 2.5% (w/v) L proline, and 0.01% (w/v) polysorbate 80 at pH 5.2. In various embodiments, the composition comprises 110 mg/mL anti-TSLP antibody, 10 mM acetate, 3.0% (w/v) L-proline, and 0.01% (w/v) polysorbate 80, at pH 5.2.

Another aspect of the disclosure is an article of manufacture comprising any one of the presently disclosed compositions, optionally, comprising about 0.5 mL to about 5 mL (e.g., about 0.5 mL to about 3 mL) of the composition.

Another aspect of the disclosure is a prefilled syringe comprising any one of the presently disclosed compositions, optionally, comprising about 0.5 mL to about 5 mL (e.g., about 0.5 mL to about 3 mL) of the composition.

Another aspect of the disclosure is a vial comprising any one of the presently disclosed compositions, optionally, comprising about 0.5 mL to about 5 mL (e.g., about 0.5 mL to about 3 mL) of the composition.

Also provided is an autoinjector containing the composition described herein, optionally, comprising about 0.5 mL to about 5 mL (e.g., about 0.5 mL to about 3 mL) of the composition. In various embodiments, the auto-injector is an Ypsomed YpsoMate®. In various embodiments, the auto-injector is disclosed in WO 2018/226565, WO 2019/094138, WO 2019/178151, WO 20120/072577, W02020/081479, WO 2020/081480, PCT/US20/70590, PCT/US20/70591, PCT/US20/53180, PCT/US20/53179, PCT/US20/53178, or PCT/US20/53176.

Another aspect of the disclosure is a method for treating an inflammatory disease in a subject comprising administering to the subject a therapeutically effective amount of the composition of any one of the preceding claims. In exemplary instances, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps and idiopathic pulmonary fibrosis (IPF). In exemplary aspects, the method comprises administering the composition at an interval of every 2 weeks or every 4 weeks. Optionally, the composition is administered for a period of at least 4 months, 6 months, 9 months, 1 year or more. In various embodiments, the inflammatory disease is asthma. In some aspects, the asthma is severe asthma, eosinophilic or non-eosinophilic asthma, or low eosinophil asthma. In exemplary instances, the subject is an adult. In alternative aspects, the subject is a child or adolescent. In exemplary instances, the administration decreases eosinophils in blood, sputum, broncheoalveolar fluid, or lungs of the subject. In some aspects, the administration shifts cell counts in the subject from a Th2 high population to a Th2 low population. In certain aspects, the administration improves one or more measures of asthma in a subject selected from the group consisting of forced expiratory volume (FEV), FEV1 reversibility, forced vital capacity (FCV), FeNO, Asthma Control Questionnaire-6 score and AQLQ(S)+12 score. In exemplary aspects, the administration improves one or more symptoms of asthma as measured by an asthma symptom diary. In various embodiments, the administration is subcutaneous or intravenous. In various embodiments, the administration is subcutaneous.

Another aspect of the disclosure is a presentation of the composition for storage or use, e.g. in a single-use vial, single-use syringe, or glass, glass-lined, or glass-coated primary container.

Another aspect of the disclosure provides the use of tezepelumab, or another human anti-TSLP monoclonal antibody or an antigen-binding portion thereof, in the manufacture of a medicament as described herein for treating a subject in need of an anti-TSLP monoclonal antibody.

Another aspect of the disclosure is a kit including a composition or article described herein together with a package insert, package label, instructions, or other labeling directing or disclosing any of the methods or embodiments disclosed herein.

Another aspect of the disclosure is method of making a stable, liquid antibody composition having a viscosity of less than about 24 cP and comprising less than about 200 mg/mL an anti-TSLP antibody, a surfactant and a buffer, said method comprising (i) combining a first solution comprising the antibody at a first concentration, acetate and proline with a buffer comprising acetate and proline, to obtain a solution comprising about 110 mg/mL to about 140 mg/mL tezepelumab, proline and acetate and (ii) adding a surfactant to the solution to achieve a final concentration of about 0.01% (w/v) ±0.005% (w/v) surfactant The viscosity of the stable, liquid composition after adding the proline is, in some aspects, less than about 20 cP. In exemplary aspects, a solution comprising about 200 mM to about 300 mM proline is combined with the first solution. In exemplary aspects, the proline is L-proline. In certain instances, the surfactant is polysorbate 80 or polysorbate 20. In exemplary aspects, the buffer is made with glacial acetic acid. In various aspects, the buffer comprises about 1 mM to about 30 mM acetate, optionally, about 5 mM to about 15 mM acetate. In some instances, the pH of the stable, liquid antibody composition is about 5.2.

In an additional aspect, provided is a method of making a stable, liquid antibody composition having a viscosity of less than about 24 cP and comprising less than about 200 mg/mL an anti-TSLP antibody, a surfactant and a buffer, said method comprising formulating the anti-TSLP antibody with a buffer comprising about 10 mM to about 20 mM acetate and about 2.7% (w/v) to about 3.3% (w/v) having a pH of about 4.9 to about 5.5, and (ii) adding a surfactant to achieve a final concentration of about 0.005% (w/v)±0.015% (w/v) surfactant. In various embodiments, the buffer is made using glacial acetic acid. In various embodiments, the buffer is titrated to pH 5.2 using sodium hydroxide.

Also provided is a solution for injection (i) comprising about 110 mg/mL to about 115 mg/mL tezepelumab, about 24 mM to about 26 mM acetate made using glacial acetic acid, about 2.4% to about 2.6% (w/v) L-proline, about 0.01% polysorbate 80, sodium hydroxide, and water for injection, (ii) having a pH of about 5.2 and a shelf-life of about 3 years. In various embodiments, provided is a prefilled syringe comprising about 1.91 mL of the stable, liquid antibody composition.

Further aspects and advantages will be apparent to those of ordinary skill in the art from a review of the following detailed description, taken in conjunction with the drawings. While the compositions, articles, and methods are susceptible of embodiments in various forms, the description hereafter includes specific embodiments with the understanding that the disclosure is illustrative, and is not intended to limit the invention to the specific embodiments described herein. For the compositions, articles, and methods described herein, optional features, including but not limited to components, compositional ranges thereof, substituents, conditions, and steps, are contemplated to be selected from the various aspects, embodiments, and examples provided herein.

### BRIEF DESCRIPTION OF THE DRAWINGS

FIG. 1A is a graph of the viscosity (cP) as a function of protein (tezepelumab) concentration (mg/mL) in formulations comprising either sucrose (circles) or proline (squares). Formulations were made at lab scale and did not comprise a surfactant during viscosity measurements. Assay temperature: 20° C.

FIG. 1B is a graph of the size exclusion chromatography (SEC) % main peak of a series of formulations comprising ˜130 mg/mL tezepelumab and either proline or sorbitol after storage at 40° C., 30° C., or 2° C.-8° C. as a function of time (months). Formulations were made at lab scale and comprised a surfactant.

FIG. 2 is a graph of the SEC % main peak of a formulation comprising tezepelumab (˜130 mg/mL) and proline (circles), proline and calcium acetate (squares), or proline and magnesium acetate (triangles) stored at 40° C. as a function of time (months). Formulations were made at lab scale and comprised a surfactant.

FIG. 3 is a graph of the SEC % main peak of a series of formulations comprising ˜110 mg/mL tezepelumab stored at 2° C. to 8° C. as a function of time (months). Two different lots of antibody (Lot A and Lot B) were used. Formulations were made at lab scale and comprised a surfactant.

FIG. 4 is a graph of the SEC % main peak of a three lots of tezepelumab (˜140 mg/mL) stored at −30° C. as a function of time (months) and stored in single use system bags were used. The lots of drug substance were made at large-scale and comprised a surfactant.

FIG. 5A is a graph of the SEC % main peak of four lots of tezepelumab (˜110 mg/mL) stored at 40° C. as a function of time (months). One sample of Lot 1 was filled into a prefilled syringe (PFS) and then stored. Samples of Lots 1-4 were filled into a vial and then stored. The lots of drug product were made at large-scale and comprised a surfactant.

FIG. 5B is a graph of the SEC % main peak of four lots of tezepelumab (˜110 mg/mL) stored at 30° C. as a function of time (months). A sample of each of Lots 1-3 was filled into a prefilled syringe (PFS) and then stored. A sample of Lot 5 was filled into a vial and then stored. The lots of drug product were made at large-scale and comprised a surfactant.

FIG. 6A is a graph of the SEC % main peak of five lots tezepelumab (˜110 mg/mL) stored at 25° C. as a function of time (months). A sample of each of Lots 1-3 was filled into a prefilled syringe (PFS) and then stored. A sample of Lots 1-5 was filled into a vial and then stored. The lots of drug product were made at large-scale and comprised a surfactant.

FIG. 6B is a graph of the SEC % main peak of five lots of tezepelumab (˜110 mg/mL) stored at 2-8° C. as a function of time (months). A sample of each of Lots 1-2 was filled into a prefilled syringe (PFS) and then stored. A sample of each of Lots 1-5 was filled into a vial and then stored. The lots of drug product were made at large-scale and comprised a surfactant.

FIGS. 7A-7B show the SEC % main peak for the proline formulations in Table 6 at 2-8° C. or 25° C. up to 6 months, or 1 month at 40° C., of storage in a prefilled syringe (FIG. 7A) or in glass vials (FIG. 7B). Formulations were made at lab scale.

FIGS. 8A-8B show the SEC % HWM species for the proline formulations in Table 6 at 2-8° C. or 25° C. up to 6 months, or 1 month at 40° C., of storage in a prefilled syringe (FIG. 8A) or in glass vials (FIG. 8B). Formulations were made at lab scale.

FIGS. 9A-9B show the CEX % main peak for the proline formulations in Table 6 at 2-8° C. or 25° C. up to 6 months, or 1 month at 40° C., of storage in a prefilled syringe (FIG. 9A) or in glass vials (FIG. 9B). Formulations were made at lab scale.

FIGS. 10A-10B show the rCE-SDS Heavy chain (HC) and light chain (LC) peak for the proline formulations in Table 6 at 2-8° C. or 25° C. up to 6 months, or 1 month at 40° C., of storage in a prefilled syringe (FIG. 10A) or in glass vials (FIG. 10B). Formulations were made at lab scale.

### DETAILED DESCRIPTION

Definitions

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The compositions and methods are contemplated to include embodiments including any combination of one or more of the additional optional elements, features, and steps further described below (including those shown in the figures), unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It should be understood that every maximum numerical limitation given throughout this specification includes as alternative aspects ranges formed with every corresponding lower numerical limitation, as if such ranges were expressly written. Every minimum numerical limitation given throughout this specification will include as alternative aspects ranges formed with every higher numerical limitation, as if such ranges were expressly written. Every numerical range given throughout this specification will include every narrower numerical range that falls within such broader numerical range, as if such narrower numerical ranges were all expressly written herein. The dimensions and values disclosed herein should be understood to include disclosure of both the recited value and the corresponding exact numerical, e.g., a value described as “about 10 mM” should be understood to include, as an alternative disclosure, “10 mM.”

All patents, publications and references cited herein are hereby fully incorporated by reference. In case of conflict between the present disclosure and incorporated patents, publications and references, the present disclosure should control.

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “asthma” as used herein refers to allergic, non-allergic, eosinophilic, and non-eosinophillic asthma.

The term “allergic asthma” as used herein refers to asthma that is triggered by one or more inhaled allergens. Such patients have a positive IgE fluorescence enzyme immunoassay (FEIA) level to one or more allergens that trigger an asthmatic response.

Typically, most allergic asthma is associated with Th2-type inflammation.

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

The term “eosinophilic asthma” as used herein refers to an asthma patient having a screening blood eosinophil count of ≥300 cells/μL or ≥250 cells/μL. In various embodiments, the blood eosinophil count is ≥300 cells/μL, ≥250 cells/μL, ≥200 cells/μL or ≥150 cells/μL. “Low eosinophilic” asthma refers to asthma patients having less than 250 cells/uL blood or serum.

The term “Th2-type inflammation” as used herein refers to a subject having a screening blood eosinophil count≥140 cells/μL and a screening total serum IgE level of >100 IU/mL (Corren et al, N Engl J Med. 22; 365(12)1088-98, 2011). A “Th2 high” asthma population or profile refers to a subject having IgE>100 IU/mL and Blood Eosinophil Count≥140 cells/μL. A “Th2 low” asthma population refers to a subject having IgE<100 IU/mL and Blood Eosinophil Count≤140 cells/μL.

An “elevated FeNO” (Fractional exhaled nitric oxide) as used herein refers to a baseline FeNO measurement greater than or equal to the median from all randomized subjects in the study. Elevated FeNO refers to FeNO levels of 24 or above.

The term “elevated serum periostin level” as used herein refers to a patient having a baseline serum periostin level greater than or equal to the median from all randomized subjects in the study. Periostin has been shown to be involved in certain aspects of allergic inflammation, including eosinophil recruitment, airway remodeling, and development of a Th2 phenotype (Li et al., Respir Res. 16(1):57, 2015).

The term “current post-bronchodilator (BD) forced expiratory volume in 1 second (FEV₁) reversibility” as used herein refers to a post-BD change in FEV₁ of ≥12% and ≥200 mL

The term “asthma exacerbation” as used herein refers to a worsening of asthma that leads to any of the following: Use of systemic corticosteroids for at least 3 days; a single depo-injectable dose of corticosteroids is considered equivalent to a 3-day course of systemic corticosteroids; for subjects receiving maintenance OCS, a temporary doubling of the maintenance dose for at least 3 days qualifies; an ED visit due to asthma that required systemic corticosteroids (as per above); an inpatient hospitalization due to asthma. Additional measures associated with asthma exacerbations are also being examined to determine effect. These include hospitalizations related to asthma exacerbations (i.e., severe asthma exacerbations), time to first asthma exacerbation, and the proportion of subjects with one or more asthma exacerbation/severe asthma exacerbation.

The term “worsening of asthma” refers to new or increased symptoms and/or signs (examination or lung function) that can be either concerning to the subject (subject-driven) or related to an Asthma Daily Diary alert (diary-driven) via the ePRO device. Asthma-worsening thresholds include: decrease in morning peak flow≥30% on at least 2 of 3 successive days compared with baseline (last 7 days of run-in), and/or a ≥50% increase in rescue medication (minimum increase of 2 or more puffs, or one new or additional nebulized β2 agonist) on at least 2 of 3 successive days compared with the average use for the previous week, and/or nocturnal awakening due to asthma requiring rescue medication use for at least 2 of 3 successive nights, and/or an increase in total asthma symptom score (the sum of daytime [evening assessment] and nighttime [morning assessment]) of at least 2 units above the screening/run-in period average (last 10 days of screening/run-in), or the highest possible score (daily score of 6), on at least 2 of 3 successive days.

The term “cytokine” as used herein refers to one or more small (5-20 kD) proteins released by cells that have a specific effect on interactions and communications between cells or on the behavior of cells, such as immune cell proliferation and differentiation. Functions of cytokines in the immune system include, promoting influx of circulating leukocytes and lymphocytes into the site of immunological encounter; stimulating the development and proliferation of B cells, T cells, peripheral blood mononuclear cells (PBMCs) and other immune cells; and providing antimicrobial activity. Exemplary immune cytokines, include but are not limited to, IL-1, IL-2, IL-3, IL-4, IL-5, IL-6, IL-7, IL-9, IL-10, IL-12, IL-13, IL-15, IL17A, IL-17F, IL-18, IL-21, IL-22, interferon (including IFN alpha, beta, and gamma), tumor necrosis factor (including TNF alpha, beta), transforming growth factor (including TGF alpha, beta), granulocyte colony stimulating factor (GCSF), granulocyte macrophage colony stimulating factor (GMCSF) and thymic stromal lymphopoietin (TSLP).

A “T helper (Th) 1 cytokine” or “Th1-specific cytokine” refers to cytokines that are expressed (intracellularly and/or secreted) by Th1 T cells, and include IFN-g, TNF-a, IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-a, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds a target antigen with greater affinity than other antigens of related proteins. It is contemplated herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-a) and the like.

The term “antibody” or “immunoglobulin” refers to the canonical tetrameric glycoprotein that consists of two substantially full-length heavy chains and two substantially full-length light chains, each comprising a variable region and a substantially full-length constant region. Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies. “Antibody” or “immunoglobulin can also refer to chimeric or CDR-grafted antibodies.

Antibody variants include antibody fragments and anti-body like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic, single-chain antibodies (scFv), single chain antibody fragments, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity, and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, single-chain antibodies (scFv), single chain antibody fragments, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Valency” refers to the number of antigen binding sites on each antibody or antibody fragment that targets an epitope. A typical full length IgG molecule, or F(ab)₂ is “bivalent” in that it has two identical target binding sites. A “monovalent’ antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following:
binding of TSLP to its receptor;
proliferation, activation, or differentiation of cells expressing TSLPR in the presence of TSLP;
inhibition of Th2 cytokine production in a polarization assay in the presence of TSLP;
dendritic cell activation or maturation in the presence of TSLP;
mast cell cytokine release in the presence of TSLP.
See, e.g., U.S. Pat. No. 7,982,016 B2, column 6 and example 8 and US 2012/0020988 A1, examples 7-10.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

Low-Viscosity Anti-TSLP Antibody Compositions

Tezepelumab has shown effectiveness at strengths ranging from 70 mg to 280 mg and the anti-TSLP antibody, in some instances, will be formulated at doses of 110 mg/mL or 140 mg/mL. Formulations with high protein concentrations may exhibit increased viscosity to a point where the functionality of the device used to administer the antibody to the patient may be negatively impacted. Similarly, the ability of a health care provider to manually inject the drug into the patient may be compromised. High viscosity can additionally be prohibitive during manufacturing. Formulations with high protein concentrations also are challenging from the standpoint of protein stability. For example, aggregation resulting in the formation of high molecular weight species (HMWS) can occur in formulations comprising high concentrations of protein. It is therefore desirable to provide a low viscosity, isotonic, liquid formulation of an anti-TSLP antibody, such as tezepelumab, suitable for parenteral administration that can be stored long term at cold temperatures (e.g., at 2-8° C. and −30° C.) or short term at room temperature (e.g., 20-25° C., for patient convenience).

Provided herein are liquid formulations (i.e., liquid compositions) suitable for parenteral administration that may be stored long term or short term comprising a high concentration of an anti-TSLP antibody (e.g., greater than about 100 mg/mL), a surfactant, proline, and a buffer. In exemplary embodiments, the anti-TSLP antibody is present in the composition at a concentration greater than about 100 mg/mL and, optionally, less than about 200 mg/mL or less than about 150 mg/mL. In some aspects, the anti-TSLP antibody is present in the composition at a concentration of about 105 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 195 mg/mL, about 196 mg/mL, about 197 mg/mL, about 198 mg/mL, about 199 mg/mL. In various aspects, the anti-TSLP antibody is present in the composition at a concentration of about 105 mg/mL to about 190 mg/mL, about 105 mg/mL to about 180 mg/mL, about 105 mg/mL to about 170 mg/mL, about 105 mg/mL to about 160 mg/mL, about 105 mg/mL to about 150 mg/mL, about 105 mg/mL to about 140 mg/mL, about 105 mg/mL to about 130 mg/mL, about 105 mg/mL to about 120 mg/mL, about 110 mg/mL to about 190 mg/mL, about 120 mg/mL to about 190 mg/mL, about 130 mg/mL to about 190 mg/mL, about 140 mg/mL to about 190 mg/mL, about 150 mg/mL to about 190 mg/mL, about 160 mg/mL to about 190 mg/mL, about 170 mg/mL to about 190 mg/mL, or about 180 mg/mL to about 190 mg/mL. In various aspects, the anti-TSLP antibody is present in the composition at a concentration of about 105 mg/mL to about 115 mg/mL or about 108 mg/mL to about 112 mg/mL, or about 130 mg/mL to about 150 mg/mL or about 135 mg/mL to about 145 mg/mL. In exemplary aspects, the anti-TSLP antibody is present in the composition at a concentration of about 110 mg/mL to about 140 mg/mL, e.g., about 110 mg/mL±10%, about 140 mg/mL±10%.

The compositions of the present disclosure comprise a surfactant. Surfactants are surface active agents that are amphipathic (having a polar head and hydrophobic tail). Surfactants preferentially accumulate at interfaces, resulting in reduced interfacial tension. Use of a surfactant can also help to mitigate formation of large proteinaceous particles. In some aspects, the surfactant present in the compositions of the present disclosure is an amphipathic and/or nonionic surfactant. Exemplary surfractants include polyoxyethylene sorbitan fatty acid esters (e.g. polysorbate 20, polysorbate 80), alkylaryl polyethers, e.g. oxyethylated alkyl phenol (e.g. Triton™ X-100), and poloxamers (e.g. Pluronics®, e.g. Pluronic® F68), and combinations of any of the foregoing, either within a class of surfactants or among classes of surfactants. Polysorbate 20 and polysorbate 80 (and optionally mixtures thereof) are particularly contemplated. The surfactant in exemplary instances is present in the composition at a concentration of less than or about 0.015% (w/v)±0.005% (w/v). For instance, the formulation may comprise about 0.005% (w/v) to about 0.015% (w/v) surfactant, e.g., about 0.005% (w/v), about 0.006% (w/v), about 0.007% (w/v), about 0.008% (w/v), about 0.009% (w/v), about 0.010% (w/v), about 0.011% (w/v), about 0.012% (w/v), about 0.013% (w/v), about 0.014% (w/v), about 0.015% (w/v). In exemplary aspects, the formulation comprises about 0.005% (w/v), 0.010% (w/v), or 0.015% (w/v) surfactant.

The compositions of the present disclosure comprise proline, e.g., L-proline, D-proline. In some aspects, the composition comprises less than about 3.0% (w/v) proline. For example, the composition comprises, in exemplary aspects, about 2.0% (w/v), about 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), or about 3.0% (w/v) proline, e.g., L-proline. For example, the composition comprises, in exemplary aspects, about 2.0% (w/v) to about 2.1% (w/v), about 2.0% (w/v) to about 2.2% (w/v), about 2.0% (w/v) to about 2.3% (w/v), about 2.0% (w/v) to about 2.4% (w/v), about 2.0% (w/v) to about 2.5% (w/v), about 2.0% (w/v) to about 2.6% (w/v), about 2.0% (w/v) to about 2.7% (w/v), about 2.0% (w/v) to about 2.8% (w/v), or about 2.0% (w/v) to about 2.9% (w/v) proline, e.g., L-proline. In various instances, the composition comprises about 2.4% (w/v) to about 2.8% (w/v) or about 2.5% (w/v) to about 2.8% (w/v) or about 2.6% (w/v) to about 2.8% (w/v) or about 2.7% (w/v) to about 2.8% (w/v). In some aspects, proline is the only amino acid present in the composition. In various embodiments, the composition comprises about 140 mM to about 280 mM proline, about 150 mM to about 250 mM proline, about 160 mM to about 240 mM proline, about 170 mM to about 230 mM proline, or about 180 to about 220 mM proline. In various embodiments, the composition comprises about 140 mM proline, about 150 mM proline, about 160 mM proline, about 170 mM proline, about 180 mM proline, about 190 mM proline, about 200 mM proline, about 210 mM proline, about 220 mM proline, about 230 mM proline, about 240 mM proline, about 250 mM proline, about 260 mM proline, about 270 mM proline, or about 280 mM proline. In exemplary instances, proline is the only amino acid present in the composition which reduces viscosity of the composition.

The composition of the present disclosure comprises a buffer. The buffer can be, for instance, an organic buffer. The buffer in some aspects, is centered at 25° C. around pH 4 to 5.5, or 4.5 to 5.5, or 4.5 to 5, for example. In various embodiments, the buffer can have a pKa within one pH unit of pH 5.0-5.2 at 25° C. One such buffer is acetic acid/acetate, having a pKa of about 4.75 at 25° C. Another such buffer is glutamic acid/glutamate, having a pKa of about 4.27 at 25° C. Other alternative buffers contemplated include buffers based on ions including succinate (pKa of 4.21 at 25° C.), propionate (pKa of 4.87 at 25° C.), malate (pKa of 5.13 at 25° C.), pyridine (pKa of 5.23 at 25° C.) and piperazine (pKa of 5.33 at 25° C.). It is contemplated that the buffer can be provided as the sodium salt (or disodium salt, as appropriate), or in the alternative as a potassium, magnesium, or ammonium salt. Buffers based on acetate, glutamate, and succinate are particularly contemplated, e.g. acetate or glutamate. In some aspects, the buffer is made with glacial acetic acid or with glutamic acid. Optionally, sodium hydroxide is added until the target pH is reached.

In exemplary aspects, the buffer is selected from the group consisting of: glutamate, histidine, and acetate. In some aspects, the buffer is acetate, and, optionally, the buffer is made with glacial acetic acid. In exemplary instances, the composition comprises about 1 mM to about 50 mM buffer, e.g., about 1 mM to about 40 mM buffer or about 1 mM to about 30 mM. In various aspects, the composition comprises about 5 mM to about 40 mM, about 10 mM to about 30 mM buffer, optionally, about 15 mM to about 30 mM buffer, about 20 mM to about 30 mM buffer, or about 10 mM to about 25 mM buffer. In exemplary aspects, the buffer is present in the composition at a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM or about 30 mM buffer. In certain embodiments, the buffer is an acetate buffer optionally made from glacial acetic acid where sodium hydroxide is added until the target pH is reached. In various instances, the composition comprises about 20 mM to about 28 mM buffer, optionally, about 23 mM to about 28 mM or about 24 mM to about 28 mM buffer (e.g., acetate). In various aspects, the composition comprises about 22 mM to about 26 mM buffer (e.g., acetate). In various aspects, the composition comprises about 24 mM to about 26 mM buffer (e.g., acetate). As described herein, in various aspects, the concentration of the buffer depends on the concentration of the anti-TSLP antibody. In various aspects, the concentration of the buffer (e.g., acetate) is about 20 mM to about 28 mM, when the concentration of the antibody is about 110 mg/mL to about 140 mg/mL. Optionally, when the concentration of the antibody is about 110 mg/mL, the composition comprises about 22 mM to about 26 mM or about 24 mM to about 26 mM buffer (e.g., acetate). Optionally, when the concentration of the antibody is about 140 mg/mL, the composition comprises about 24 mM to about 26 mM or about 25 mM to about 26 mM buffer (e.g., acetate).

In various embodiments, the composition comprises 110 mg/mL anti-TSLP antibody with 10 mM acetate, 3.0% (w/v) L-proline, and 0.01% (w/v) polysorbate 80, ata final pH of 5.2. In various embodiments, the composition comprises 110 mg/mL anti-TSLP antibody, formulated in 24 mM acetate, 2.5% (w/v) L proline, and 0.01% (w/v) polysorbate 80 at pH 5.2. In various embodiments, the composition comprises 110 mg/mL tezepelumab formulated in 24 mM acetate, 2.5% (w/v) L proline, and 0.01% (w/v) polysorbate 80 at pH 5.2.

In exemplary aspects, the composition of the present disclosure may comprise additional components. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In alternative aspects, the composition consists essentially of or consists of the anti-TSLP antibody, a surfactant, proline, and a buffer. In exemplary instances, the composition of the present disclosure does not comprise more than 0.001% (w/v) of a sugar or citrate, optionally, wherein the sugar is a disaccharide, e.g., trehalose and sucrose.

In alternative aspects, the composition of the present disclosure is a liquid. In certain aspects, the liquid has a pH which is less than about 6.0, optionally, less than about 5.5. In some aspects, the pH is about 4.5 to about 5.5 or about 4.8 to about 5.4, e.g., about 4.8, about 4.9, about 5.0, about 5.1, about 5.2, about 5.3, about 5.4. In some aspects, the pH is about 4.9, 5.2, or 5.4. In some aspects, the composition is characterized by a reduced viscosity, relative to liquid composition not comprising proline. In exemplary instances, the composition is characterized by a viscosity of less than about 24 centiPoise (cP) at 20° C. when the concentration of the anti-TSLP antibody is less than 155 mg/mL, optionally, −6 cP when the concentration of the anti-TSLP antibody is about 110 mg/mL or about 15 cP when the concentration of the anti-TSLP antibody is about 140 mg/mL. In certain aspects, the composition is characterized by a viscosity of about 5 cP to about 20 cP, e.g., about 5 cP to about 15 cP, about 5 cP to about 10 cP, about 10 cP to about 20 cP, about 15 cP to about 20 cP, or about 5 cP, about 6 cP, about 7 cP, about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, when the concentration of the anti-TSLP antibody is less than 155 mg/mL (e.g., about 110 mg/mL, about 140 mg/mL). In exemplary aspects, the composition has a viscosity that is about 15 cP±5 cP when the concentration of the antibody is about 100 mg/mL to about 180 mg/mL. Unless noted otherwise, all viscosities disclosed herein refers to a viscosity measured using a rotational viscometer at 20° C. and at a shear rate of about 1000 l/s.

In exemplary aspects, the composition is intended for subcutaneous administration to a subject, and thus the composition is isotonic with the intended site of administration. For example, the osmolality of the composition is in some aspects, in a range of about 270 to about 350 mOsm/kg, or about 285 to about 345 mOsm/kg, or about 300 to about 315 mOsm/kg. For example, if the solution is in a form intended for administration parenterally, it can be isotonic with blood (about 300 mOsm/kg osmolality). In exemplary aspects, the aqueous pharmaceutical formulation has an osmolality in a range of about 200 mOsm/kg to about 500 mOsm/kg, or about 225 mOsm/kg to about 400 mOsm/kg, or about 250 mOsm/kg to about 350 mOsm/kg.

The composition of the present disclosure is advantageously suitable for long-term or short-term storage. In exemplary aspects, the composition is suitable for long- or short-term storage at frozen or refrigerated temperatures or at higher temperatures. Accordingly, the compositions of the present disclosure may be stored at temperatures below 0° C. (e.g., about −80° C. to about −10° C., about −60° C. to about −20° C., or about −30° C.) or at temperatures of about 1° C. to about 10° C. (e.g., about 2° C. to about 8° C.). Optionally, the storage at these temperatures (below 10° C.) may be a long-term storage, e.g., at least 6 months, at least 12 months, at least 18 months, at least 24 months, at least 30 months, at least 36 months. The compositions of the present disclosure may be stored at room temperature (e.g., about 20° C. to about 30° C., about 23° C. to about 27° C., about 25° C., or about 30° C.). In various aspects, the compositions of the present disclosure may be stored at temperatures above room temperature (e.g., greater than 30° C. (e.g., about 35° C. to about 45° C., about 40° C.).

In various aspects, the composition of the present disclosure is highly stable and can endure long term storage at refrigerated or frozen temperatures. The composition of the present disclosure is highly stable as a liquid or as a solid. Optionally, less than about 5% (e.g., less than about 4%, less than about 3%, less than about 2%, less than about 1%) of the therapeutic protein is degraded after about 1 month to about 3 months of storage at about −40° C. to about −20° C., (e.g., about −35° C., about −30° C., about −25° C., about −20° C.). In some aspects, less than about 5% (e.g., less than about 4%, less than about 3%, less than about 2%, less than about 1%) of the therapeutic protein is degraded after 6 months or 12 months of storage at about −40° C. to about −20° C. as determined by SEC and optionally, the therapeutic protein is contained in glass vials or syringes. In some aspects, less than about 5% (e.g., less than about 4%, less than about 3%, less than about 2%, less than about 1%) of the therapeutic protein is degraded after 24 months or 36 months of storage at about −40° C. to about −20° C. as determined by SEC, and optionally, the therapeutic protein is contained in glass vials or syringes. In various embodiments, more than 95% of the therapeutic protein is intact after 24 months of storage at about −40° C. to about −20° C. in glass vials or syringes, as determined by SEC. In some aspects, less than about 5% (e.g., less than about 4%, less than about 3%, less than about 2%, less than about 1%) of the antibody in the composition of the present disclosure is degraded after about 24 months of storage at about −40° C. to about −20° C. as determined by SEC, optionally, wherein less than 5% (e.g., less than about 4%, less than about 3%, less than about 2%, less than about 1%) of the antibody is degraded after 36 months of storage at about −40° C. to about −20° C. Optionally, less than about 5% (e.g., less than about 4%, less than about 3%, less than about 2%, less than about 1%) of the therapeutic protein is degraded after about 1 month to about 3 months of storage at about 2° C. to about 8° C., (e.g., about 2° C., about 3° C., about 4° C., about 5° C., about 6° C., about 7° C., about 8° C.). In some aspects, less than about 5% (e.g., less than about 4%, less than about 3%, less than about 2%, less than about 1%) of the therapeutic protein is degraded after 6 months or 12 months of storage at about 2° C. to about 8° C. as determined by SEC and optionally, the therapeutic protein is contained in glass vials or syringes. In some aspects, less than about 5% (e.g., less than about 4%, less than about 3%, less than about 2%, less than about 1%) of the therapeutic protein is degraded after 24 months or 36 months of storage at about 2° C. to about 8° C. as determined by SEC, and optionally, the therapeutic protein is contained in glass vials or syringes. In various embodiments, more than 95% of the therapeutic protein is intact after 24 months of storage at about 2° C. to about 8° C. in glass vials or syringes, as determined by SEC. In some aspects, less than about 5% (e.g., less than about 4%, less than about 3%, less than about 2%, less than about 1%) of the antibody in the composition of the present disclosure is degraded after about 24 months of storage at about 2° C. to about 8° C. as determined by SEC, optionally, wherein less than 5% (e.g., less than about 4%, less than about 3%, less than about 2%, less than about 1%) of the antibody is degraded after 36 months of storage at about 2° C. to about 8° C.

In various aspects, the composition of the present disclosure is highly stable and can endure long term storage at room temperatures. Optionally, less than about 5% (e.g., less than about 4%, less than about 3%, less than about 2%, less than about 1%) of the therapeutic protein is degraded after about 1 month to about 3 months of storage at about 23° C. to about 27° C., (e.g., about 23° C., about 24° C., about 25° C., about 26° C., about 27° C.). In various aspects, less than 5% (e.g., less than about 4%, less than about 3%, less than about 2%, less than about 1%) of the antibody is degraded after at least 2 weeks (optionally, after at least 1 month, after at least 2 months, after at least 3 months, after at least 4 months, after at least 5 months or after at least 6 months) of storage at about room temperature (e.g., 25° C.), as determined by SEC. In some aspects, less than about 5% (e.g., less than about 4%, less than about 3%, less than about 2%, less than about 1%) of the therapeutic protein is degraded after 6 months to 12 months of storage at about 23° C. to about 27° C. as determined by SEC, and optionally, the therapeutic protein is contained in glass vials or syringes. In various embodiments, more than 95% of the therapeutic protein is intact after 24 months of storage at about 23° C. to about 27° C. in glass vials or syringes, as determined by SEC In some aspects, less than about 5% (e.g., less than about 4%, less than about 3%, less than about 2%, less than about 1%) of the antibody in the composition of the present disclosure is degraded after about 24 months of storage at about 23° C. to about 27° C. as determined by SEC optionally, wherein less than 5% (e.g., less than about 4%, less than about 3%, less than about 2%, less than about 1%) of the antibody is degraded after 36 months of storage at about 23° C. to about 27° C.

In various aspects, the composition of the present disclosure is highly stable and can endure short term storage at higher temperatures, e.g., temperatures greater than room temperature. Optionally, less than about 5% (e.g., less than about 4%, less than about 3%, less than about 2%, less than about 1%) of the therapeutic protein is degraded after about 1 month to about 3 months of storage at about 28° C. to about 32° C., (e.g., about 28° C., about 29° C., about 30° C., about 31° C., about 32° C.). In some aspects, less than about 5% (e.g., less than about 4%, less than about 3%, less than about 2%, less than about 1%) of the therapeutic protein is degraded after 6 months or 12 months of storage at about 28° C. to about 32° C. as determined by SEC, and optionally, the therapeutic protein is contained in glass vials or syringes. In various embodiments, more than 95% of the therapeutic protein is intact after 24 months of storage at about 28° C. to about 32° C. in glass vials or syringes, as determined by SEC. In some aspects, less than about 5% (e.g., less than about 4%, less than about 3%, less than about 2%, less than about 1%) of the antibody in the composition of the present disclosure is degraded after about 24 months of storage at about 28° C. to about 32° C. as determined by SEC, optionally, wherein less than 5% (e.g., less than about 4%, less than about 3%, less than about 2%, less than about 1%) of the antibody is degraded after 36 months of storage at about 28° C. to about 32° C. In exemplary aspects, less than about 5% (e.g., less than about 4%, less than about 3%, less than about 2%, less than about 1%) of the therapeutic protein is degraded after 6 months of storage at about 30° C. as determined by SEC.

In various aspects, the composition of the present disclosure is highly stable and can endure short term storage under stressed storage conditions. Optionally, less than about 5% (e.g., less than about 4%, less than about 3%, less than about 2%, less than about 1%) of the therapeutic protein is degraded after about 1 week or after about 2 weeks or after about 1 month to about 3 months of storage at about 38° C. to about 42° C., (e.g., about 38° C., about 39° C., about 40° C., about 41° C., about 42° C.).

In various aspects, the composition of the present disclosure is highly stable and can endure mixed or combined storage conditions. Optionally, less than 5% (e.g., less than about 4%, less than about 3%, less than about 2%, less than about 1%) of the antibody is degraded after 24 months of storage at about 2° C. to about 8° C. followed by 2 weeks or more of storage at about 25° C., as determined by SEC. In various aspects, less than 5% of the antibody is degraded after 2 weeks of storage at about 25° C. as determined by SEC. Optionally, less than 5% of the antibody is degraded after about 24 months to about 36 months of storage at about 2° C. to about 8° C. followed by about 4 weeks to about 8 weeks of storage at about 25° C., as determined by SEC. In various instances, less than 5% of the antibody is degraded after about 24 months to about 36 months of storage at 2° C. to 8° C. followed by at least 2 weeks or at least about 1 month or at least about 2 months of storage at about room temperature (e.g., 25° C.), as determined by SEC.

In exemplary aspects of the disclosure, the composition is provided for storage or use, e.g. in a single-use vial, single-use syringe, or glass, glass-lined, or glass-coated primary container. In exemplary aspects, the composition is provided in a single use system bag or a polycarbonate carboy for frozen storage. In alternative aspects, the composition is contained in glass vials or syringes for storage, e.g., long-term storage, at about 2° C. to about 8° C. or storage at higher temperatures (e.g., about 25° C., about 30° C., about 40° C.).

In exemplary instances, the composition is provided for use in a delivery system which is off-the-shelf and/or designed for self-administration. In exemplary aspects, the composition is provided in a prefilled syringe or an autoinjector, a pen injector, a dual-chamber pen, and the like. Such products are known in the art and are commercially available. See, e.g., Shire, Steven, Monoclonal Antibodies: Meeting the Challenges in Manufacturing, Formulation, Delivery and Stability of Final Drug Product, Chapter 8: Development of delivery device technology to deal with the challenges of highly viscous mAb formulations at high concentration, Woodhead Publishing, Cambridge, UK, pages 153-162 (2015). In exemplary aspects, the composition is provided for use in an YpsoMate™ autoinjection, an YpsoMate™ 2.25 autoinjector, or a VarioJect™ (YpsoMed, Burgdorf, Switzerland). Other autoinjectors include, e.g., SelfDose™ Patient-Controlled Injector, BD Physioject™ disposable autoinjector, Autoject® II Syringe Injector (Owen Mumford, Oxfordshire, UK). In various embodiments, the autoinjector is an Ypsomed YpsoMate® autoinjector. Additional autoinjectors contemplated for in the methods are disclosed in International Patent Publications WO 2018/226565, WO 2019/094138, WO 2019/178151, WO 20120/072577, WO2020/081479, WO 2020/081480, and International Patent Application Nos. PCT/US20/70590, PCT/US20/70591, PCT/US20/53180, PCT/US20/53179, PCT/US20/53178, and PCT/US20/53176, incorporated by reference herein.

The composition of the present disclosure can be suitable for administration by any acceptable route, including parenteral, and specifically subcutaneous. For example, the subcutaneous administration can be to the upper arm, upper thigh, or abdomen. Other routes include intravenous, intradermal, intramuscular, intraperitoneal, intranodal and intrasplenic, for example. The subcutaneous route is preferred.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored in a ready-to-use form.

The composition of the present disclosure comprises an anti-TSLP antibody. In exemplary embodiments, the anti-TSLP antibody specifically binds to a TSLP polypeptide as set forth in amino acids 29-159 of SEQ ID NO: 2. Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+ progenitor cells. Swedin et al., Pharmacol Ther 169: 13-34 (2017). TSLP signals through a heterodimeric receptor consisting of the interleukin (IL)-7 receptor alpha (IL-7Rα) chain and a common y chain-like receptor (TSLPR) (Pandey, Nat Immunol. 1:59-64, 2000; Park, J Exp Med. 192:659-669, 2000).

Human TSLP mRNA (Brightling et al., J Allergy Clin Immunol 121:5-10 quiz 1-2 (2008); Ortega et al., NEJM 371:1198-1207 (2014)) and protein levels (Ortega et al., (2014), supra) are increased in the airways of asthmatic individuals compared to controls, and the magnitude of this expression correlates with disease severity. Brightling et al, (2008), supra. Recent studies have demonstrated association of a single nucleotide polymorphism in the human TSLP locus with protection from asthma, atopic asthma and airway hyperresponsiveness, suggesting that differential regulation of TSLP gene expression might influence disease susceptibility. (To et al., BMC Public Health 12: 204 (2012); XOLAIR® (omalizumab): Highlights of Prescribing Information 2016. (at https://www.gene.com/download/pdf/xolair_prescribing.pdf.); Bleecker et al., The Lancet 388: 2115-2127 (2016). These data suggest that targeting TSLP may inhibit multiple biological pathways involved in asthma.

Earlier non-clinical studies of TSLP suggested that after TSLP is released from airway epithelial cells or stromal cells, it activates mast cells, dendritic cells, and T cells to release Th2 cytokines (e.g., IL-4/13/5). Recently published human data demonstrated a good correlation between tissue TSLP gene and protein expression, a Th2 gene signature score, and tissue eosinophils in severe asthma. Therefore, an anti-TSLP target therapy may be effective in asthmatic patients with Th2-type inflammation (Shikotra et al, J Allergy Clin Immunol. 129(1):104-11, 2012).

Data from other studies suggest that TSLP may promote airway inflammation through Th2 independent pathways such as the crosstalk between airway smooth muscle and mast cells (Allakhverdi et al, J Allergy Clin Immunol. 123(4):958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al, Clin Exp Allergy. 39(1):89-100, 2009). These data and other emerging evidence suggest that blocking TSLP may serve to suppress multiple biologic pathways including but not limited to those involving Th2 cytokines (IL-4/13/5).

It is contemplated that antibodies specific for TSLP are useful in the treatment of asthma, including severe asthma, eosinophlic asthma, no-eosinophilic/low-eosinophilic and other forms of asthma described herein.

Specific binding agents such as antibodies and antibody variants or fragments that bind to their target antigen, e.g., TSLP, are useful in the methods of the invention. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the disclosure are human antibodies and antibody variants (including antibody fragments) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

Antibodies and antibody variants (including antibody fragments) useful in the present methods comprise an anti-TSLP antibody comprising (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

Also contemplated is an antibody or antibody variant comprising (A) a light chain variable domain selected from the group consisting of: (i) a sequence of amino acids at least 80% (e.g., about 85%, about 90%, about 95%, greater than 95%) identical to SEQ ID NO:12; (ii) a sequence of amino acids encoded by a polynucleotide sequence that is at least 80% (e.g., about 85%, about 90%, about 95%, greater than 95%) identical to SEQ ID NO:11; (iii) a sequence of amino acids encoded by a polynucleotide that hybridizes under moderately stringent conditions to the complement of a polynucleotide consisting of SEQ ID NO:11; and (B) a heavy chain variable domain selected from the group consisting of: (i) a sequence of amino acids that is at least 80% (e.g., about 85%, about 90%, about 95%, greater than 95%) identical to SEQ ID NO:10; (ii) a sequence of amino acids encoded by a polynucleotide sequence that is at least 80% (e.g., about 85%, about 90%, about 95%, greater than 95%) identical to SEQ ID NO:9; (iii) a sequence of amino acids encoded by a polynucleotide that hybridizes under moderately stringent conditions to the complement of a polynucleotide consisting of SEQ ID NO:9; or (C) a light chain variable domain of (A) and a heavy chain variable domain of (A), wherein the antibody or antibody variant specifically binds to a TSLP polypeptide as set forth in amino acids 29-159 of SEQ ID NO:2.

In exemplary instances, the anti-TSLP antibody comprises a heavy chain comprising the amino acid sequence of SEQ ID NO: 13, a light chain comprising the amino acid sequence of SEQ ID NO: 14, or a heavy chain comprising the amino acid sequence of SEQ ID NO: 13 and a light chain comprising the amino acid sequence of SEQ ID NO: 14.

Tezepelumab is an exemplary anti-TSLP antibody having (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

Tezepelumab also comprises:
(A) a light chain variable domain selected from the group consisting of:
   (i) a sequence of amino acids at least 80% identical to SEQ ID NO:12;
   (ii) a sequence of amino acids encoded by a polynucleotide sequence that is at least 80% identical to SEQ ID NO:11;
   (iii) a sequence of amino acids encoded by a polynucleotide that hybridizes under moderately stringent conditions to the complement of a polynucleotide consisting of SEQ ID NO:11; and
(B) a heavy chain variable domain selected from the group consisting of:
   (i) a sequence of amino acids that is at least 80% identical to SEQ ID NO:10;
   (ii) a sequence of amino acids encoded by a polynucleotide sequence that is at least 80% identical to SEQ ID NO:9;
   (iii) a sequence of amino acids encoded by a polynucleotide that hybridizes under moderately stringent conditions to the complement of a polynucleotide consisting of SEQ ID NO:9; or
(C) a light chain variable domain of (A) and a heavy chain variable domain of (B).

Other exemplary anti-TSLP antibodies are known in the art. See, e.g., International Patent Application Publication Nos. WO2017/042701, WO2016/142426, WO2010/017468, U.S. Patent Application Publication No. US2012/0020988, and U.S. Pat. No. 8,637,019. In exemplary aspects, the anti-TSLP antibody is an antibody disclosed in one of these publications.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody. In exemplary aspects, the anti-TSLP antibody is an IgG2 antibody.

In various embodiments, the anti-TSLP antibody variant is selected from the group consisting of a diabody, a triabody, a tetrabody, a Fab fragment, single domain antibody, scFv, wherein the dose is adjusted such that the binding sites to be equimolar to the those dosed by bivalent antibodies. In exemplary aspects, both binding sites of the antibody have identical binding to TSLP.

It is contemplated that the antibody or antibody variant is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

Consistent with the foregoing, in some aspects, the composition of the present disclosure comprises about 110 mg/mL to about 140 mg/mL anti-TSLP antibody (e.g., tezepelumab), about 0.01% (w/v) ±0.005% (w/v) polysorbate 80, about 2.4% (w/v) to about 2.8% (w/v) L-proline, and about 20 mM to about 28 mM acetate, wherein the viscosity of the composition is less than about 20 cP (e.g., 15 cP) and the pH is less than about 5.5, optionally, about 5.2. Optionally, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In exemplary instances, the composition comprises about 110 mg/mL of an anti-TSLP antibody, e.g., tezepelumab, 0.01% (w/v) polysorbate 80, about 2.4% (w/v) to about 2.8% (w/v) L-proline, and about 20 mM to about 28 mM acetate (e.g., about 22 mM to about 26 mM, about 24 mM to about 26 mM), wherein the composition has a pH of about 5.2, wherein the anti-TSLP antibody optionally comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In alternative instances, the composition comprises about 140 mg/mL of an anti-TSLP antibody, e.g., tezepelumab, 0.01% (w/v) polysorbate 80, about 2.5% (w/v) to about 2.8% (w/v) L-proline, and about 20 mM to about 28 mM acetate (e.g., about 24 mM to about 26 mM, about 25 mM to about 26 mM), wherein the composition has a pH of about 5.2, wherein the anti-TSLP antibody optionally comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In various embodiments, the composition comprises 110 mg/mL anti-TSLP antibody, 24 mM acetate, 2.5% (w/v) L proline, and 0.01% (w/v) polysorbate 80 at pH 5.2. In various embodiments, the composition comprises 110 mg/mL anti-TSLP antibody, 10 mM acetate, 3.0% (w/v) L-proline, and 0.01% (w/v) polysorbate 80, at pH 5.2.

Methods of Making

Methods of making the composition of the present disclosure are further provided herein. Accordingly, methods of making a stable, liquid composition having a viscosity of less than about 24 cP and comprising less than about 200 mg/mL (about 100 mg/mL to about 180 mg/mL) an anti-TSLP antibody, a surfactant and a buffer are further provided. In exemplary embodiments, the method comprises: (i) combining a first solution comprising the antibody at a first concentration, acetate and proline with a buffer comprising acetate and proline, to obtain a solution comprising about 110 mg/mL to about 140 mg/mL tezepelumab, proline and acetate and (ii) adding a surfactant to the solution to achieve a final concentration of about 0.01% (w/v)±0.005% (w/v) surfactant. In exemplary aspects, the stable, liquid composition comprises about 110 mg/mL or about 140 mg/mL of the anti-TSLP antibody. In some aspects, the viscosity of the stable, liquid composition with the proline is reduced relative to a liquid composition without the proline. For example, in some instances, the viscosity of the stable, liquid formulation is less than about 20 cP. In exemplary aspects, a solution comprising about 200 mM to about 300 mM proline (e.g., about 220 mM to about 280 mM, about 245 mM to about 275 mM, about 255 mM to about 265 mM, or about 260 mM) is combined with the first solution. Optionally, the proline is L-proline. In some aspects, the surfactant is polysorbate 80 or polysorbate 20. In exemplary instances, the surfactant is polysorbate 80 and the final concentration of PS80 is about 0.01% (w/v). In exemplary aspects, the buffer is made with glacial acetic acid and optionally, the target pH is reached upon addition of sodium hydroxide. In various instances, the buffer comprises about 1 mM to about 30 mM acetate, optionally, about 5 mM to about 15 mM acetate In various aspects, the pH of the buffer is the same as the pH of the stable, liquid composition. In exemplary instances, the pH of the stable, liquid composition is about 5.2. In exemplary embodiments, the anti-TSLP antibody is tezepelumab.

Articles of Manufacture, Syringes, and Vials

The present disclosure provides an article of manufacture comprising any one of the presently disclosed compositions, optionally, comprising about 0.5 mL to about 5 mL (e.g., about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 5 mL, about 1.5 mL to about 5 mL, about 2 mL to about 5 mL, about 2.5 mL to about 5 mL, about 3 mL to about 5 mL, about 3.5 mL to about 5 mL, about 4 mL to about 5 mL, about 4.5 mL to about 5 mL) of the composition. In various aspects, the article of manufacture comprises about 0.64 mL to 2.09 mL of any one of the presently disclosed compositions. Optionally, the article of manufacture comprises about 1.91 mL of any one of the presently disclosed compositions. Optionally, the composition comprises about 100 mg/mL to about 280 mg/mL anti-TSLP antibody (e.g., tezepelumab). In various aspects, the composition comprises about 110 mg/mL to about 140 mg/mL anti-TSLP antibody (e.g., tezepelumab), about 0.01% (w/v)±0.005% (w/v) polysorbate 80, about 2.4% (w/v) to about 2.8% (w/v) L-proline, and about 20 mM to about 28 mM acetate, wherein the viscosity of the composition is less than about 20 cP (e.g., 15 cP) and the pH is less than about 5.5, optionally, about 5.2.

The present disclosure also provides a prefilled syringe (PFS) comprising any one of the presently disclosed compositions, optionally, comprising about 0.5 mL to about 5 mL (e.g., about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 5 mL, about 1.5 mL to about 5 mL, about 2 mL to about 5 mL, about 2.5 mL to about 5 mL, about 3 mL to about 5 mL, about 3.5 mL to about 5 mL, about 4 mL to about 5 mL, about 4.5 mL to about 5 mL) of the composition. In various aspects, the PFS comprises about 0.64 mL to 2.09 mL of any one of the presently disclosed compositions. Optionally, the PFS comprises about 1.91 mL of any one of the presently disclosed compositions. Optionally, the composition comprises about 100 mg/mL to about 280 mg/mL anti-TSLP antibody (e.g., tezepelumab). In various aspects, the composition comprises about 110 mg/mL to about 140 mg/mL anti-TSLP antibody (e.g., tezepelumab), about 0.01% (w/v) ±0.005% (w/v) polysorbate 80, about 2.4% (w/v) to about 2.8% (w/v) L-proline, and about 20 mM to about 28 mM acetate, wherein the viscosity of the composition is less than about 20 cP (e.g., 15 cP) and the pH is less than about 5.5, optionally, about 5.2.

Also provided is a vial comprising any one of the presently disclosed compositions, optionally, comprising about 0.5 mL to about 5 mL (e.g., about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 5 mL, about 1.5 mL to about 5 mL, about 2 mL to about 5 mL, about 2.5 mL to about 5 mL, about 3 mL to about 5 mL, about 3.5 mL to about 5 mL, about 4 mL to about 5 mL, about 4.5 mL to about 5 mL) of the composition. In various aspects, the vial comprises about 0.64 mL to 2.09 mL of any one of the presently disclosed compositions. Optionally, the vial comprises about 1.91 mL of any one of the presently disclosed compositions. Optionally, the composition comprises about 100 mg/mL to about 280 mg/mL anti-TSLP antibody (e.g., tezepelumab). In various aspects, the composition comprises about 110 mg/mL to about 140 mg/mL anti-TSLP antibody (e.g., tezepelumab), about 0.01% (w/v)±0.005% (w/v) polysorbate 80, about 2.4% (w/v) to about 2.8% (w/v) L-proline, and about 20 mM to about 28 mM acetate, wherein the viscosity of the composition is less than about 20 cP (e.g., 15 cP) and the pH is less than about 5.5, optionally, about 5.2.

Kits

The present disclosure also provides a kit including a composition described herein together with a package insert, package label, instructions, or other labeling directing or disclosing any of the methods or embodiments disclosed herein. In certain embodiments, the present disclosure provides kits for producing a single-dose administration unit. In certain embodiments of this disclosure, kits containing single and multi-chambered prefilled syringes (e.g., liquid syringes) are included.

Methods of Use

The present disclosure also provides the use of tezepelumab, or another human anti-TSLP monoclonal antibody or an antigen-binding portion thereof, in the manufacture of a medicament as described herein for treating a subject in need of an anti-TSLP monoclonal antibody.

Contemplated herein are methods for treating an inflammatory disease in a subject. In exemplary embodiments, the methods comprise administering to the subject a therapeutically effective amount of a composition comprising greater than about 100 mg/mL of an anti-TSLP antibody, a surfactant, proline, and a buffer. In certain embodiments, the composition is a sterile pharmaceutical composition.

As used herein, “inflammatory disease” refers to a medical condition involving abnormal inflammation caused by the immune system attacking the body's own cells or tissues, which may result in chronic pain, redness, swelling, stiffness, and damage to normal tissues. Inflammatory diseases include, for example, asthma, chronic peptic ulcer, tuberculosis, periodontitis, sinusitis, active hepatitis, ankylosing spondylitis, rheumatoid arthritis, chronic obstructive pulmonary disease (COPD), Crohn's disease, ulcerative colitis, osteoarthritis, atherosclerosis, systemic lupus erythematosus, atopic dermatitis, eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps, idiopathic pulmonary fibrosis (IPF), and the like. In exemplary aspects, the inflammatory disease is asthma, atopic dermatitis, or COPD. In exemplary aspects, the inflammatory is asthma and, in some instances, the asthma is severe asthma, eosinophilic asthma, non-eosinophilic asthma, or low eosinophil asthma. Surprisingly, it was found herein that treatment with an anti-TSLP antibody is effective at reducing asthma symptoms in a no eosinophil/low eosinophil population as it is in a high eosinophil population. In some aspects, the method reduces the frequency of asthma exacerbation in a subject.

Also contemplated herein are methods of treating asthma in a subject having a Th2 high asthma profile or a Th2 low asthma profile. It is contemplated that a TSLP antagonist that inhibits binding of the TSLP protein to its receptor complex will effectively treat a low eosinophil asthma population as the antibody described herein. Similarly, it is contemplated that a TSLP antagonist that inhibits binding of TSLP to its receptor complex will be effective in treating Th2 low asthma populations.

Provided herein is a method of treating a patient having low eosinophil asthma comprising administering the composition of the present disclosure. Also contemplated is a method for treating a subject having asthma characterized by a low Th2 profile comprising administering the composition of the present disclosure comprising an anti-TSLP antibody. In various embodiments, the antibody is tezepelumab or another anti-TSLP antibody described in the art. Exemplary anti-TSLP antibodies include antibodies described in WO 2017/042701, WO 2016/142426, WO 2010/017468, US20170066823, US20120020988 and U.S. Pat. No. 8,637,019, incorporated herein by reference. Also contemplated are methods for treating chronic obstructive pulmonary disease (COPD) in a subject comprising administering an anti-TSLP antibody or antibody variant.

It is contemplated that the subject is human. The subject may be an adult, an adolescent or a child.

Therapeutic antibody (or antibody variant) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the composition provides a dose of the anti-TSLP antibody or antibody variant within the range of about 70 mg to about 280 mg per daily dose. For example, the dose provided may be about 70 mg, 210 mg or 280 mg. In various embodiments, the composition comprising the anti-TSLP antibody or antibody variant may be administered at a dose of 70, 80, 90, 100, 110, 120, 130, 140, 150, 160, 10, 180, 190, 200, 210, 220, 230, 240, 250, 260, 270 or 280 mg per dose. These concentrations may be administered as a single dosage form or as multiple doses. The above doses are given every two weeks or every four weeks. In various embodiments, the anti-TSLP antibody or antibody variant is administered at a single dose of 70 mg every two weeks or every four weeks. In various embodiments, the anti-TSLP antibody or antibody variant is administered at a single dose of 210 mg every two weeks or every four weeks. In various embodiments, the composition comprising greater than about 100 mg/mL anti-TSLP antibody is administered to the subject at an interval of every two weeks or every four weeks.

For antibody variants, the amount of antibody variant should be such that the number of TSLP binding sites that are in the dose have an equimolar number of TSLP binding sites to canonical bivalent antibody described above.

It is contemplated that the composition of the present disclosure comprising the anti-TSLP antibody or antibody variant is administered every 2 weeks or every 4 weeks for a period of at least 4 months, 6 months, 9 months, 1 year or more. In various embodiments, the administration is subcutaneous or intravenous. In various embodiments, the administration is subcutaneous.

Treatment with the anti-TSLP antibody or antibody variant is contemplated to decrease eosinophils in blood, sputum, broncheoalveolar fluid, or lungs of the subject. It is also contemplated that the administration shifts cell counts in the subject from a Th2 high population to a Th2 low population. It is further contemplated that administration of the anti-TSLP antibody improves one or more measures of asthma in a subject selected from the group consisting of forced expiratory volume (FEV), FEV1 reversibility, forced vital capacity (FCV), FeNO, Asthma Control Questionnaire-6 score and AQLQ(S)+12 score.

Measures of diagnosis and assessment of asthma include the following:

Airway inflammation evaluated using a standardized single-breath Fraction of Exhaled Nitric Oxide (FeNO)(American Thoracic Society; ATS, Am J Respir Crit Care Med. 171(8):912-30, 2005) test. For example, subjects inhale to total lung capacity through the NIOX MING® Airway Inflammation Monitor and then exhale for 10 seconds at 50 mL/sec (assisted by visual and auditory cues).

Spirometry is performed according to ATS/European Respiratory Society (ERS) guidelines (Miller et al, Eur Respir J. 26(1):153-61, 2005). For example, multiple forced expiratory efforts (at least 3 but no more than 8) are performed at each spirometry session and the 2 best efforts that meet ATS/ERS acceptability and reproducibility criteria are recorded. The best efforts will be based on the highest FEV1. The maximum FEV1 of the 2 best efforts will be used for the analysis. Both the absolute measurement (for FEV1 and FVC) and the percentage of predicted normal value will be recorded using appropriate reference values. The highest FVC will also be reported regardless of the effort in which it occurred (even if the effort did not result in the highest FEV1).

Post-bronchodilator (Post-BD) spirometry testing is assessed after the subject has performed pre-BD spirometry. Maximal bronchodilation is induced using a SABA such as albuterol (90 μg metered dose) or salbutamol (100 μg metered dose) or equivalent with a spacer device for a maximum of 8 total puffs (Sorkness et al, J Appl Physiol. 104(2):394-403, 2008). The highest pre- and post-BD FEV1 obtained after 4, 6, or 8 puffs is used to determine reversibility and for analysis. Reversibility algorithm is as follows:

% Reversibility=(post-BD FEV1−pre-BD FEV1)×100/pre-BD FEV1

Home peak flow testing for peak expiratory flow rate (PEFR) is performed twice daily, in the morning upon awakening and in the evening prior to bedtime using a peak flow meter from the morning of Visit 2 (Week −4) through Week 64. When possible, ambulatory lung function measurements should be taken at least 6 hours after the last dose of SABA rescue medication.

The Asthma Daily Diary includes the following daily assessments: asthma symptoms; inhalations of rescue medication; nighttime awakening due to asthma requiring rescue medication use, asthma-related activity limitations, asthma-related stress, and background medication compliance. The Asthma Daily Diary is completed each morning and evening. There will be triggers in the ePRO device to alert the subjects to signs of worsening of asthma.

The Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV1 (Juniper et al, Oct 1999). The ACQ-6 is a shortened version of the ACQ that omits the FEV1 measurement from the original ACQ score. Questions are weighted equally and scored from 0 (totally controlled) to 6 (severely uncontrolled). The mean ACQ score is the mean of the responses. Mean scores of ≤0.75 indicate well-controlled asthma, scores between 0.75 and 1.5 indicate partly-controlled asthma, and a score >1.5 indicates uncontrolled asthma (Juniper et al, Respir Med. 100(4):616-21, 2006). Individual changes of at least 0.5 are considered to be clinically meaningful (Juniper et al, Respir Med. 99(5):553-8, 2005).

The Asthma Quality of Life Questionnaire, Standardized (AQLQ[S])+12 (AQLQ(S)+12) is a 32-item questionnaire that measures the HRQoL experienced by asthma patients (Juniper et al, Chest. 115(5):1265-70, May 1999). The questionnaire comprises 4 separate domains (symptoms, activity limitations, emotional function, and environmental stimuli). Subjects are asked to recall their experiences during the previous 2 weeks and to score each of the 32 questions on a 7-point scale ranging from 7 (no impairment) to 1 (severe impairment). The overall score is calculated as the mean response to all questions. The 4 individual domain scores (symptoms, activity limitations, emotional function, and environmental stimuli) are the means of the responses to the questions in each of the domains. Individual improvement in both the overall score and individual domain scores of 0.5 has been identified as a minimally important change, with score changes of ≥1.5 identified as large meaningful changes (Juniper et al, J Clin Epidemiol. 47(1):81-7, 1994).

Improvement in asthma may be measured as one or more of the following: reduction in AER (annualized exacerbation rate), reduction in hospitalizations/severe exacerbations for asthma, change from baseline (increase) in time to first asthma exacerbation (following onset of treatment with anti-TSLP antibody), decrease relative to placebo in proportion of subjects with one or more asthma exacerbations or severe exacerbations over the course of treatment, e.g., 52 weeks, change from baseline (increase) in FEV1 and FVC (pre-broncholdilator and post-bronchodilator), change from baseline (decrease) in blood or sputum eosinophils (or lung eosinophils if biopsy or BAL fluid obtained), change from baseline (decrease) in FeNO, change from baseline (decrease) in IgE, improvement in asthma symptoms and control as measured by PROs including ACQ and variants, AQLQ and variants, SGRQ, and asthma symptom diaries, change (decrease) in use of rescue medications, decrease in use of systemic corticosteroids, decrease in Th2/Th1 cell ratio in blood. Most/all these measures should be in total population and subpopulations including hi and low eosinophils (Greater than or equal to 250 is high; less than 250 is low), allergic and non-allergic, Th2 hi and low, Periostin hi and low (compared to median value), and FeNO hi and low (greater than or equal to 24 or less than 24).

The treatment also improves one or more symptoms of asthma as measured by an asthma symptom diary. Symptoms include, but are not limited to, daytime and nighttime symptom frequency and severity, activity avoidance and limitation, asthma-related stress and fatigue as well as rescue asthma medication use), and other measures of asthma control as measured by the Asthma Control Questionnaire omitting FEV₁ (ACQ-6).

In various embodiments, treatment with the composition of the present disclosure comprising the anti-TSLP antibody delays the time to an asthma exacerbation compared to a subject not receiving the anti-TSLP antibody.

Also contemplated in the present disclosure is the administration of multiple agents, such as an antibody composition in conjunction with a second agent as described herein, including but not limited to an anti-inflammatory agent or asthma therapy.

However, it is contemplated that, in various embodiments, the administration reduces frequency of or levels of co-administered therapy in the subject. Exemplary co-administered therapy include, but are not limited to, inhaled corticosteroids (ICS), long-acting β2 agonist (LABA), leukotriene receptor antagonists [LTRA], long-acting anti-muscarinics [LAMA], cromones, short-acting β2 agonist (SABA), and theophylline or oral corticosteroids. In various embodiments, the administration eliminates the need for corticosteroid therapy.

The following examples are provided for illustration and are not intended to limit the scope of the invention.

### EXAMPLES

Throughout the examples presented herein, the following abbreviations are used: DF, diafiltration; PS80, polysorbate 80; PS20, polysorbate 20; SEC, size exclusion chromatography, CEX, cation exchange chromatography, rCE, reduced capillary electrophoresis, F#, formulation number. Additionally, throughout these examples, the composition of the DF buffer used to make the final formulation comprising tezepelumab, as well as estimated concentrations of the components of the final formulation, are provided. The final concentrations of certain components of the final formulations analyzed (e.g., for stability, viscosity, optionally, after storage) differ from the concentrations of the DF or dialysis buffer, depending on the presence or absence of a counterion. Without a counterion, formulations have low ionic strength. In such instances, acetate co-concentrates with tezepelumab, such that final formulations comprise a higher concentration of acetate, relative to the concentration of the DF or dialysis buffer. For example, use of a DF buffer comprising 10 mM acetate leads to about 20 mM to about 28 mM (e.g., about 25 mM) acetate in a formulation (pH 5.2) comprising 110 mg/mL tezepelumab when neither the DF buffer nor the final formulation comprises a counterion (e.g., HCI) and thus is of low ionic strength. Similarly, a DF buffer comprising 10 mM acetate leads to about 20 mM to about 28 mM (e.g., about 25 mM) acetate a formulation (pH 5.2) comprising 140 mg/mL tezepelumab, without a counterion (e.g., HCl). When a counterion (e.g., HCl) is present, acetate does not co-concentrate with tezepelumab, and therefore the acetate concentration of the DF buffer and the acetate concentration of the final composition are generally equivalent. Additionally, excipients can be volumetrically excluded, or may be impacted by non-specific interactions. For instance, in a 110 mg/mL tezepelumab formulation, the proline concentration may be up to about 16.67% lower than what is indicated in the DF buffer, and in a 140 mg/mL tezepelumab formulation, the proline concentration may be up to about 10% to about 13.3% lower than what is indicated in the DF buffer. In view of the foregoing, throughout the following examples concentrations of the components of the final formulations are provided, taking into consideration the above described excipient exclusion and acetate co-concentration effects.

### Example 1

This example describes an exemplary method of producing a high concentration tezepelumab formulation.

High concentration formulations of tezepelumab were made using a method comprising a lab-scale tangential flow filtration (TFF) system. In this method, an initial low-concentration solution containing 70 mg/mL tezepelumab was subjected to a complete buffer exchange via diafiltration (DF) using a diafiltration buffer. Unless noted otherwise in the following examples, the diafiltration buffer comprised about 10 mM acetate and about 3% (w/v) L-proline. Diafiltration buffers were prepared using glacial acetic acid titrated to the DF buffer pH using sodium hydroxide. The pH of the diafiltration buffer varied depending on the target pH of the formulation. Following the buffer exchange step, the tezepelumab solution was concentrated to a tezepelumab concentration that was equal to or higher than the target tezepelumab concentration. If necessary, the concentrated tezepelumab solution was then diluted to the target tezepelumab concentration using a dilution buffer. Unless noted otherwise in the following examples, the dilution buffer comprised the same composition of the diafiltration buffer and comprised about 10 mM acetate and about 3% (w/v) L-proline, wherein the target pH was 5.2, unless noted otherwise. After the dilution step, a surfactant was added. In some instances, the surfactant was polysorbate 80 (PS80) and in some cases PS80 was added to each formulation at a final PS80 concentration of 0.01% (w/v). In other instances, the surfactant was polysorbate 20 (PS20) and PS20 was added to each formulation at a final concentration of 0.004% (w/v) to 0.015% (w/v).

The high concentration formulations were tested for viscosity, stability upon storage by a range of assays used to assess product quality, or a combination thereof. Viscosity was measured using a rotational viscometer at a temperature of about 20° C. to about 25° C., and the reported viscosity values are at a shear rate of about 900 l/s to about 1000 l/s. Unless noted otherwise, viscosity was measured in the absence of a surfactant. For stability, samples of each formulation were filled into containers and then stored for up to 36 months at a temperature of about −30° C. to about 40° C. (e.g., 36 months at about −30° C, 24 months at about 2° C. to about 8° C., 6 months at about 25° C., 2 months at about 30° C., 1-3 months at about 40° C. Samples were tested via size exclusion chromatography (SEC) to determine the stability of the formulation at various storage time points. Percentage of the main peak for formulations was reported and reflected the amount of tezepelumab (in monomer form) that remained after the indicated storage period.

### Example 2

This example describes an initial evaluation of the effects that different excipients have on formulation viscosity.

To evaluate the effects that sucrose and L-proline have on formulation viscosity, as a function of protein (tezepelumab) concentration, two series of tezepelumab formulations comprising one of these excipients were made with varying concentrations of tezepelumab. The tezepelumab concentrations of each series ranged from ˜50 mg/mL to >200 mg/mL. The proline-containing formulations were made as essentially described in Example 1, wherein the DF buffer comprised about 10 mM acetate and 3% (w/v) L-proline, and had a pH of 4.5 (titrated with NaOH). No surfactants were added to the proline-containing formulations. Because the initial low concentration solutions containing 70 mg/mL tezepelumab comprised sucrose, it was not required to carry out a buffer exchange step via diafiltration in order to make the sucrose-containing formulations. The sucrose-containing formulations were made by concentrating the initial low concentration solutions to a higher concentration followed by diluting with a dilution buffer comprising about 10 mM acetate, about 9.0% (w/v) sucrose, pH 5.2. The dilution buffer was made using glacial acetic acid titrated to pH 5.2 using sodium hydroxide. No surfactants were added to the sucrose-containing formulations

A sample of each formulation of each series was tested for viscosity, as essentially described in Example 1, and the results are provided below in Table 1.

TABLE 1

| | Tezepelumab | Viscosity |
|---|---|---|
| | concentration | at 20° C. |
| | (mg/mL) | (cP) |
| Proline | 51.54 | 2.41 |
| Sucrose | 52.17 | 3.96 |
| Proline | 66.08 | 3.3 |
| Sucrose | 72.36 | 5.11 |
| Proline | 109.98 | 6.68 |
| Sucrose | 110.82 | 12.82 |
| Sucrose | 137.27 | 29 |
| Proline | 141.09 | 16.29 |
| Sucrose | 150.02 | 40.41 |
| Proline | 151.02 | 23.99 |
| Sucrose | 171.34 | 79.29 |
| Proline | 188.19 | 74.58 |
| Sucrose | 193.07 | 184.56 |
| Proline | 208 | 124 |
| Sucrose | 221.05 | 448.44 |

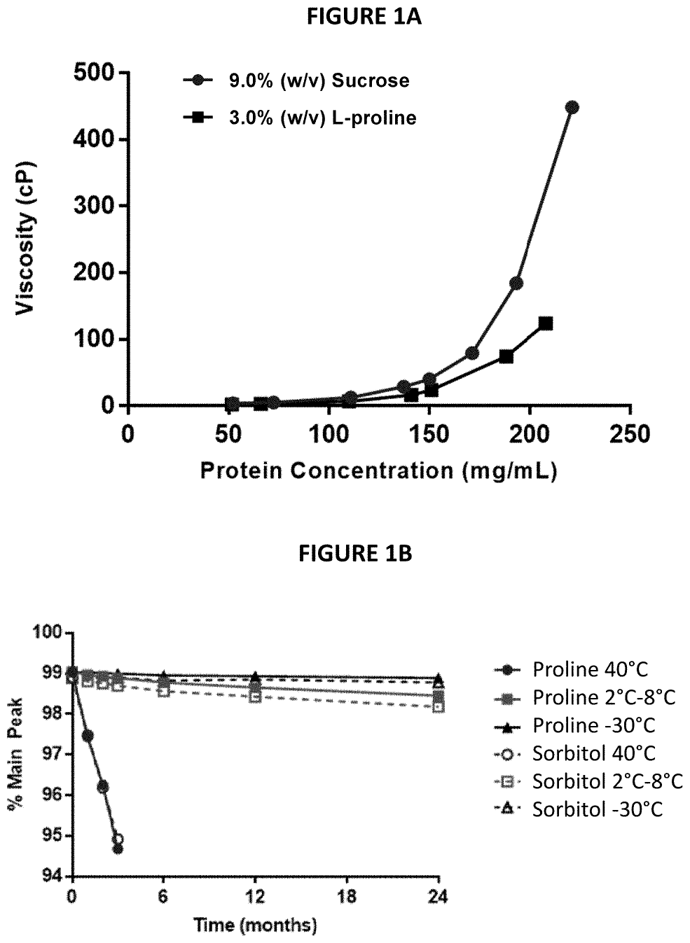

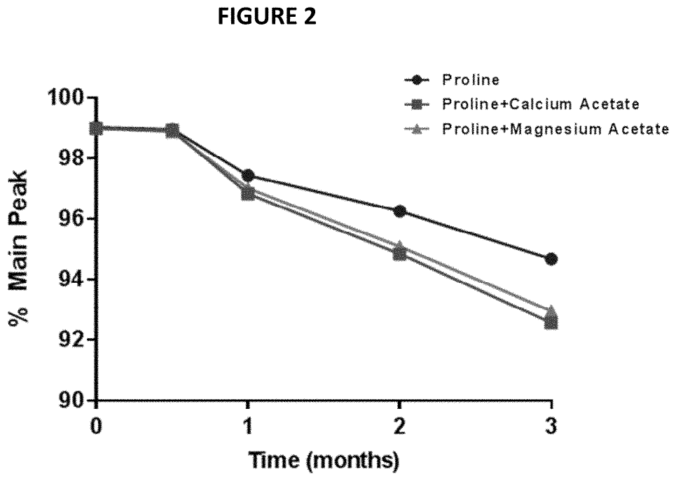

The results are also graphically represented in FIG. 1A. This figure provides a graph plotting the viscosity of the proline-containing formulations as a function of protein (tezepelumab) concentration. The viscosity of each sucrose-containing formulation is plotted on the graph for comparison. As shown in FIG. 1A, the viscosity of the sucrose formulations increased dramatically when tezepelumab concentrations exceeded 100 mg/mL, whereas the viscosity curve is shifted when tezepelumab is formulated with L-proline. Formulations comprising proline and tezepelumab at a concentration within the range of about 110 mg/mL to about 180 mg/mL exhibited a viscosity that was about half the viscosity of the sucrose formulation comprising a similar tezepelumab concentration.

This example demonstrated that proline-containing formulations exhibit significantly lower viscosities, relative to sucrose-containing formulations.

### Example 3

This example describes the effect of different excipients on the viscosity and stability of high concentration tezepelumab formulations.

Proline demonstrated a significant viscosity-reducing effect on high concentration formulations of tezepelumab. However, its effect on protein stability was not well understood. Since sucrose formulations could not be used as a control in high concentration stability studies due to difficulty in preparing material, the stability of a proline formulation was compared to that of a formulation comprising sorbitol, another commonly used excipient.

High concentration formulations of tezepelumab at ˜130 mg/mL were made as essentially described in Example 1. For the sorbitol-containing formulation, the DF buffer comprised about 5% (w/v) sorbitol and about 10 mM acetate. For the proline-containing formulation, the DF buffer comprised about 3% (w/v) L-proline and about 10 mM acetate. Each DF buffer was titrated to pH 4.6 with sodium hydroxide. The dilution buffer was the same as the DF buffer except for pH. To each formulation, PS20 was added at a final concentration of 0.01% (w/v).

Samples of each formulation were tested for viscosity as essentially described in Example 1. It was observed that the proline-containing formulation exhibited a lower solution viscosity, compared to the formulation comprising sorbitol. At 25° C., the viscosity of the proline-containing formulation was 10.2 cP compared to the sorbitol formulation which demonstrated a viscosity of 15.3 cP under the same conditions.

Samples of each formulation were tested for stability as essentially described in Example 1 under one of three storage conditions: (A) in glass syringes for up to 3 months at 40° C., (B) in glass syringes for about 24 months at 2° C.-8° C., or (C) in glass syringes for up to 24 months at 30° C. Size exclusion chromatography (SEC) was performed to monitor the physical stability (i.e. aggregation) of tezepelumab. The proline-containing formulation demonstrated acceptable stability across a range of storage temperatures, as compared to a sorbitol control. The results are shown in FIG. 1B. As shown in FIG. 1B, the physical stability of proline-containing tezepelumab formulations was as good as the sorbitol-containing formulations. More than 98% of the main peak was present after 24 months of storage at frozen or at refrigerated temperatures.

### Example 4

This example describes the effect of excipient salts in proline-containing tezepelumab formulations.

The advantages that salts impart on high concentration antibody formulations have been shown by others. It has been hypothesized that the salt acts as a shield to interrupt the protein-protein (antibody-antibody) interactions that lead to aggregation. Salts also are believed to decrease viscosity. To study whether the addition of salts improved the viscosity-lowering effect of L-proline, three formulations comprising 2% to 3% L-proline and 130 mg/mL tezepelumab were made with or without an excipient salt. The salts tested in this study included calcium acetate (25 mM) and magnesium acetate (25 mM). Each formulation was made as essentially described in Example 1 and the DF buffer used to make each formulation is shown in Table 2. PS20 was added to each formulation to a final concentration of 0.01% (w/v).

TABLE 2

| | Proline | | |
|---|---|---|---|
| Salt | % (w/v) | Acetate | pH |
| 25 mM calcium acetate | 2 | 15 mM | 5.0 |
| 25 mM magnesium acetate | 2 | 15 mM | 5.0 |
| None | 3 | 10 mM | 4.6 |

DF buffers were titrated to noted pH with NaOH.

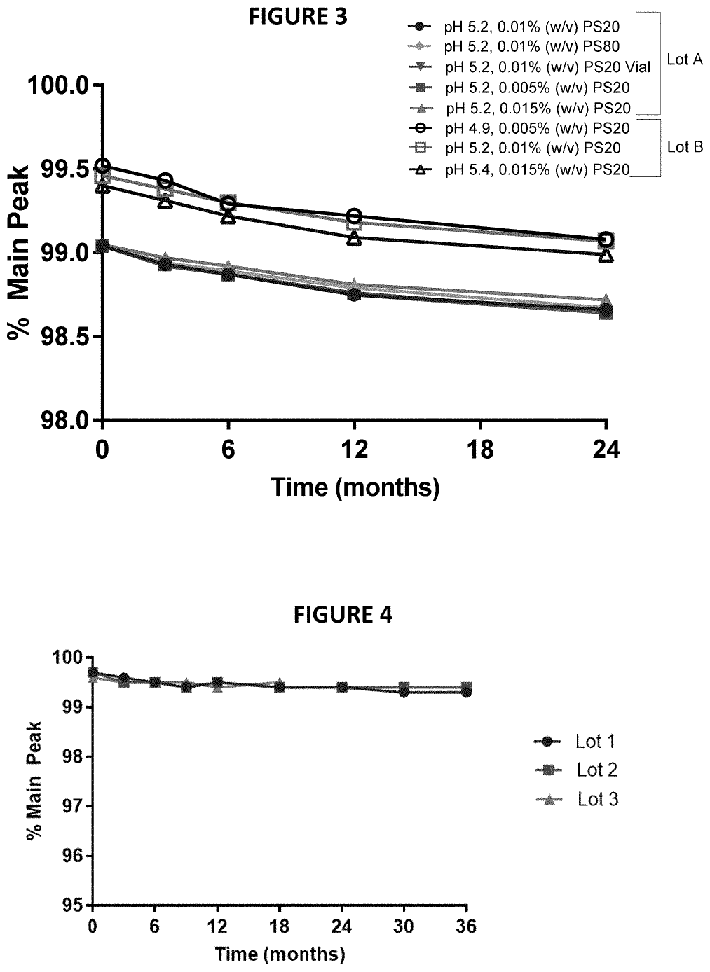

A sample of each formulation was stored in glass syringes for up to 3 months at 40° C. Size exclusion chromatography (SEC) was performed at 0, 0.5, 1, 2, and 3 months of storage to monitor the physical stability (i.e. aggregation) of tezepelumab. The results are shown in FIG. 2. As shown in FIG. 2, the physical stability (i.e. aggregation) of proline-containing tezepelumab formulations was reduced in the presence of calcium acetate or magnesium acetate, relative to the formulation containing no salt. After 3 months of storage, the formulation comprising neither calcium acetate nor magnesium acetate exhibited more than 95% of the main peak, whereas the formulations comprising one of the salts exhibited less than 94% of the main peak, indicating that more 5% of the antibody had destabilized or degraded.

Visual inspections of each formulation were also carried out. Each of the formulations comprising calcium acetate and magnesium acetate were more turbid or cloudy compared to the formulation comprising proline without any salt.

The viscosity of a sample of each formulation was measured as essentially described in Example 1, and it was demonstrated that the addition of a salt (calcium acetate and magnesium acetate) decreased viscosity.

Though the addition of salt reduced the viscosity of the high concentration formulations of tezelpelumab, the salt addition reduced the stability of the tezepelumab formulation. The results of this study were surprising and unexpected, given that the addition of salts to high concentration antibody formulations has been known to reduce protein aggregation.

### Eample 5

This example demonstrates the effect of surfactant and pH has on stability of different tezepelumab formulations.

A first series of high concentration tezepelumab formulations comprising 110 mg/mL tezepelumab was made as essentially described in Example 1. The DF buffer used to make each formulation is described in Table 3. The tezepelumab was sourced from one of two lots (Lot A or Lot B) as indicated. A surfactant, either polysorbate 20 (PS20) or polysorbate 80 (PS80), was added to the formulation at a concentration ranging from about 0.005% (w/v) to about 0.015% (w/v), as described in Example 1. The pH of the formulations varied from 4.9 to 5.4.

TABLE 3

| Estimated Final Formulation* | DF Buffer Composition |
|---|---|
| 110 mg/mL tezepelumab, Lot A, | 3% (w/v) L-proline, |
| 2.75% (w/v) ±0.25%(w/v) L-proline, 21 | 10 mM acetate, pH 4.5 |
| mM ± 1 mM acetate, pH 5.2, 0.01% | |
| (w/v) PS20 | |
| 110 mg/mL tezepelumab, Lot A, | 3% (w/v) L-proline, 1 |
| 2.75% (w/v) ±0.25%(w/v) L-proline, 21 | 0 mM acetate, pH 4.5 |
| mM ± 1 mM acetate, pH 5.2, 0.01% | |
| (w/v) PS80 | |
| 110 mg/mL tezepelumab, Lot A, | 3% (w/v) L-proline, |
| 2.75% (w/v) ±0.25%(w/v) L-proline, 21 | 10 mM acetate, pH 4.5 |
| mM ± 1 mM acetate, pH 5.2, 0.01% | |
| (w/v) PS20 | |
| 110 mg/mL tezepelumab, Lot A, | 3% (w/v) L-proline, |
| 2.75% (w/v) ±0.25%(w/v) L-proline, 21 | 10 mM acetate, pH 4.5 |
| mM ± 1 mM acetate, pH 5.2, 0.005% | |
| (w/v) PS20 | |
| 110 mg/mL tezepelumab, Lot A, | 3% (w/v) L-proline, |
| 2.75% (w/v) ±0.25%(w/v) L-proline, 21 | 10 mM acetate, pH 4.5 |
| mM ± 1 mM acetate, pH 5.2, 0.015% | |
| (w/v) PS20 | |
| 110 mg/mL tezepelumab, Lot B, | 3% (w/v) L-proline, |
| 2.75% (w/v) ±0.25%(w/v) L-proline, 21 | 15 mM acetate, pH 4.1 |
| mM ± 1 mM acetate, pH 4.9, 0.005% | |
| (w/v) PS20 | |
| 110 mg/mL tezepelumab, Lot B, | 3% (w/v) L-proline, |
| 2.75% (w/v) ±0.25%(w/v) L-proline, 21 | 10 mM acetate, pH 4.5 |
| mM ± 1 mM acetate, pH 5.2, 0.01% | |
| (w/v) PS20 | |
| 110 mg/mL tezepelumab, Lot B, | 3% (w/v) L-proline, |
| 2.75% (w/v) ±0.25%(w/v) L-proline, 21 | 10 mM acetate, pH 5.0 |
| mM ± 1 mM acetate, pH 5.4, 0.015% | |
| (w/v) PS20 | |

PS20 or PS80 was added to the final concentration noted. Storage in glass syringe unless marked with *. Buffer was titrated to final pH with NaOH.

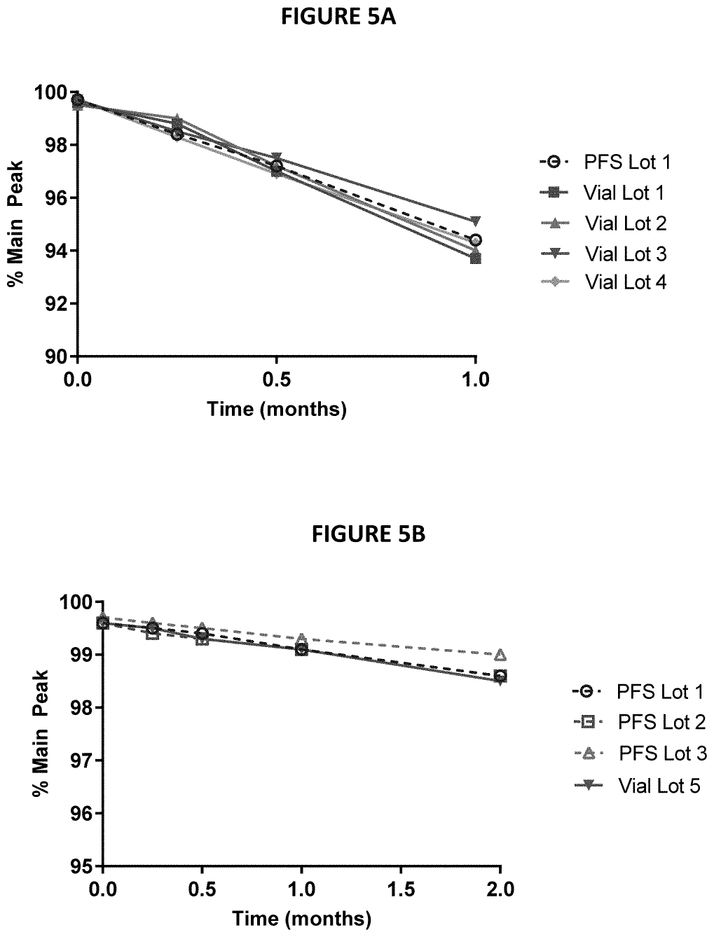

Samples of each formulation were filled into containers (glass syringe or vial) and stored at 2-8° C. for up to 24 months. SEC was carried out to determine the stability of each formulation at 0, 3, 6, 12, and 24 months of storage at 2-8° C. FIG. 3 shows the % main peak of tezepelumab for the indicated formulations. As shown in FIG. 3, each formulation comprising PS20 or PS80 across a range of pHs, demonstrated protein stability after 24 months of storage. Differences in the starting level of % main peak among the formulations may have been due to the lot variability of tezepelumab used.

### Example 6

This example demonstrates the stability of tezepelumab formulations stored at frozen temperatures.

Three lots of tezepelumab were made at large scale, each comprising about 140 mg/mL tezepelumab. Briefly, following cell culture and purification, material is diafiltered into the final formulation by ultrafiltration/diafiltration (UF/DF). The buffer used during diafiltration is 10 mM acetate (from acetic acid), 260 mM L-Proline, titrated to pH 4.5 with sodium hydroxide. Following buffer exchange, the material is over-concentrated to 180 mg/mL and diluted to 140 mg/mL tezepelumab using 10 mM acetate (from acetic acid), 260 mM L-Proline, titrated to pH 4.5 with sodium hydroxide. The final material is prepared by adding polysorbate 80 to achieve the final concentration of 0.01% (w/v). Lastly, the material is filtered and stored long term at −30° C.

A description of each is described in Table 4.

TABLE 4

| Lot | Estimated Final Formulation* | DF Buffer Composition |
|---|---|---|
| 1 | 140 mg/mL tezepelumab, 2.65% (w/v) ± | 3% (w/v) L-proline, 10 mM |
| | 0.05% (w/v) L-proline, 24 mM ± 1 mM | acetate, pH 4.5 |
| | acetate, pH 5.2, 0.01% (w/v) PS80 | |
| 2 | 140 mg/mL tezepelumab, 2.65% (w/v) ± | 3% (w/v) L-proline, 10 mM |
| | 0.05% (w/v) L-proline, 24 mM ± 1 mM | acetate, pH 4.5 |
| | acetate, pH 5.2, 0.01% (w/v) PS80 | |
| 3 | 140 mg/mL tezepelumab, 2.65% (w/v) ± | 3% (w/v) L-proline, 10 mM |
| | 0.05% (w/v) L-proline, 24 mM ± 1 mM | acetate, pH 4.5 |
| | acetate, pH 5.2, 0.01% (w/v) PS80 | |

PS80 was added to the final concentration noted. DF buffer was titrated to target pH with NaOH.

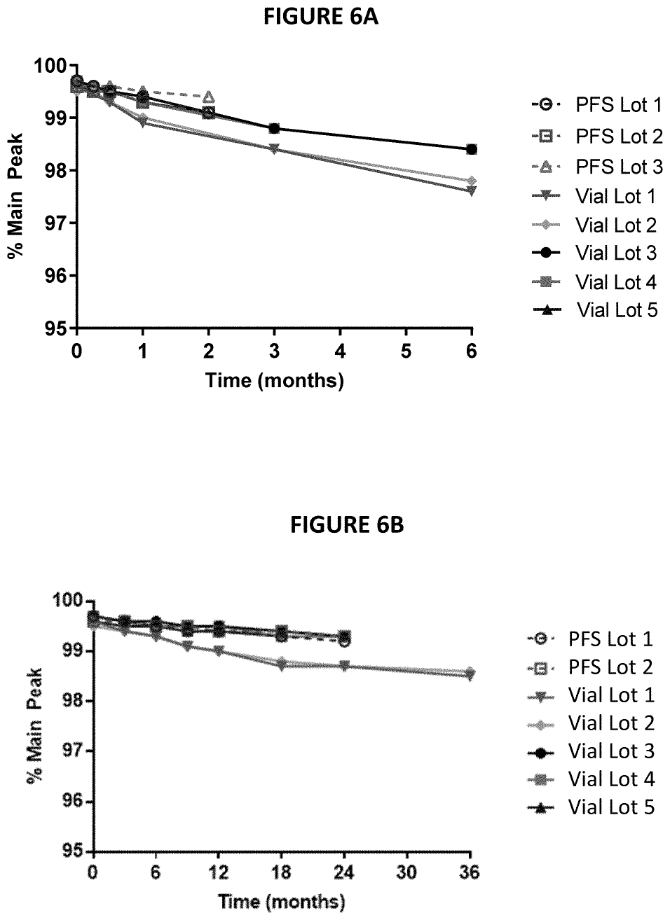

Samples of each were filled into a container (single use system (SUS) bag) and stored at −30° C. for up to 36 months. SEC was carried out to determine the stability of each formulation after 0, 3, 6, 9, 12, 18, 24, 30 and 36 months of storage. FIG. 4 shows the main peak of tezepelumab for each lot. As shown in FIG. 4, greater than 99% of the main peak was present after 36 months of storage at −30° C. for each lot. The tezepelumab lots demonstrated exceptional stability after storage at −30° C. These results were surprising and unexpected, because proline is not known as a cryoprotectant. Also, it was surprising that additional excipients (e.g., sucrose, sorbitol) were not needed to achieve the desired stability at −30° C.

### Example 7

This example demonstrates the stability of different lots of a tezepelumab formulation after storage at various times and temperatures.

Several lots of tezepelumab were made at large scale, each comprising about 110 mg/mL tezepelumab. Briefly, bulk tezepelumab comprising 140 mg/mL tezepelumab stored at −30° C. is thawed and diluted to 110 mg/mL tezepelumab using 10 mM acetate (from acetic acid), 3% (w/v) L-proline, 0.01% (w/v) polysorbate 80, titrated to 5.2 using sodium hydroxide. After dilution, material is filtered and filled into glass vials or syringes.

A concentrated tezepelumab solution comprising 140 mg/mL tezepelumab was diluted to 110 mg/mL tezepelumab using 10 mM acetate and 3.0% (w/v) proline, PS80 was added for a final concentration of 0.01% (w/v). Each formulation had a final pH of 5.2.

In a first series of studies, a volume (1.91 mL) of a tezepelumab formulation of a lot was placed into a glass prefilled syringe (PFS) having a volume capacity of 2.25 mL and stored in the horizontal orientation under different conditions as summarized in Table 5.

In another series of studies, a volume (0.64 mL to 2.09 mL, e.g., 1.91 mL) of a tezepelumab formulation from a lot was placed into a 5-cc glass vial, with a 13 mm seal and 13 mm stopper. The filled vial was stored under different conditions as summarized in Table 6.

Collectively, several lots of the tezepelumab formulation were stored at a variety of storage times and temperatures and these conditions are summarized in Table 7.

TABLE 5

| | Maximum | Storage | |
|---|---|---|---|
| | Storage Time | Temperature | |
| Study # | (months) | (° C.) | FIG. |
| 1 | 1 | 40 | 5A |
| 2 | 2 | 30 | 5B |
| 3 | 6 | 25 | 6A |
| 4 | 36 | 2-8 C | 6B |
| 5 | 36 | −30 | 4 |

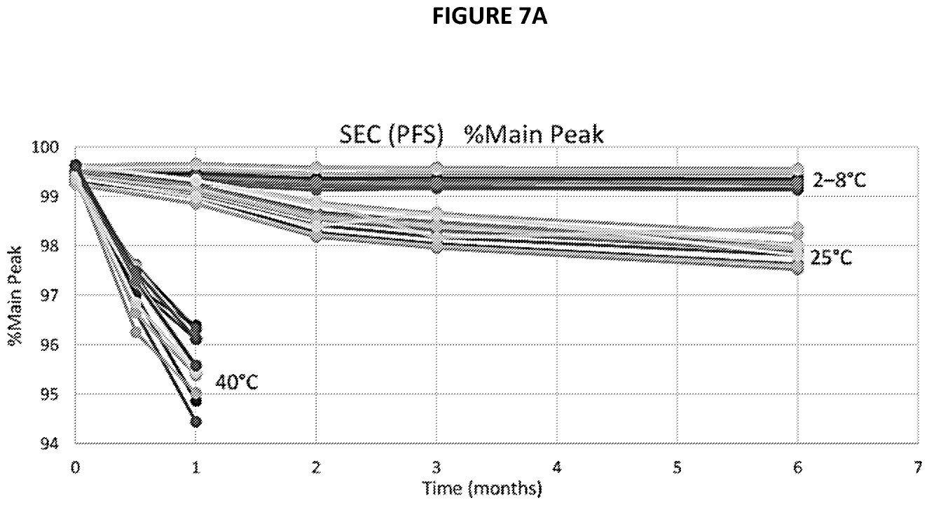

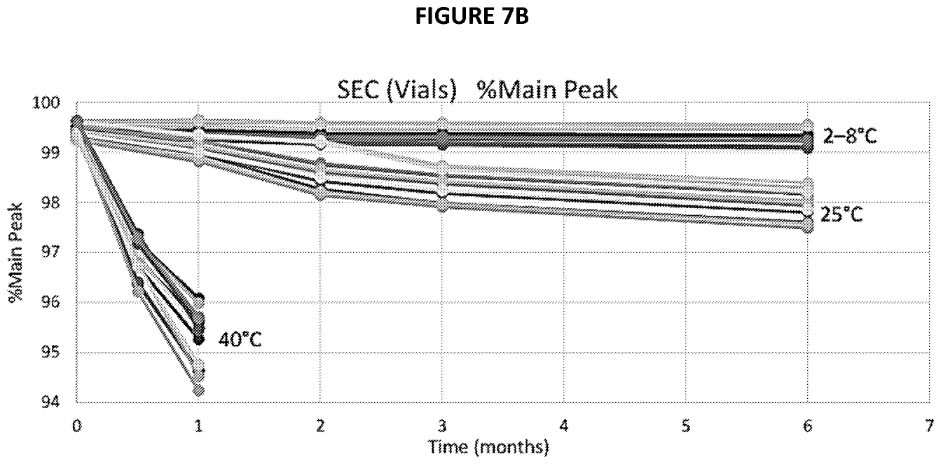

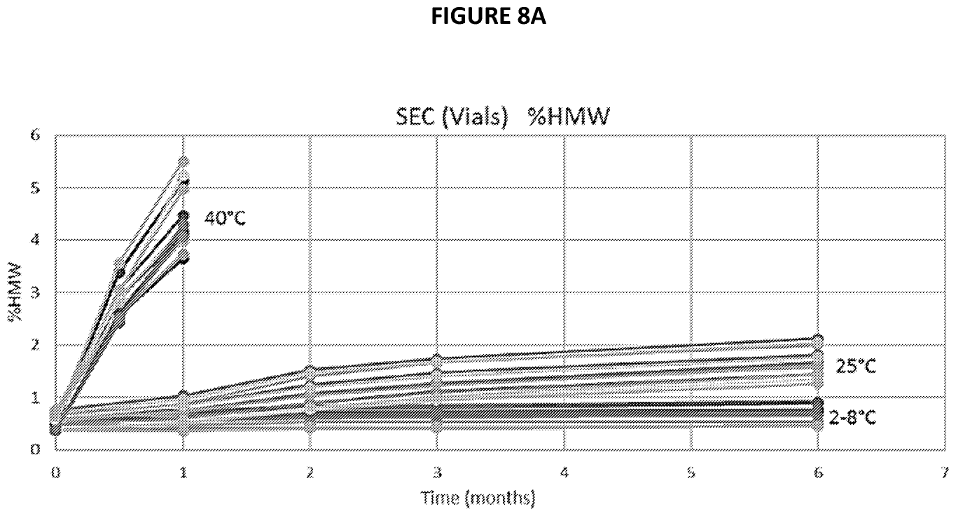

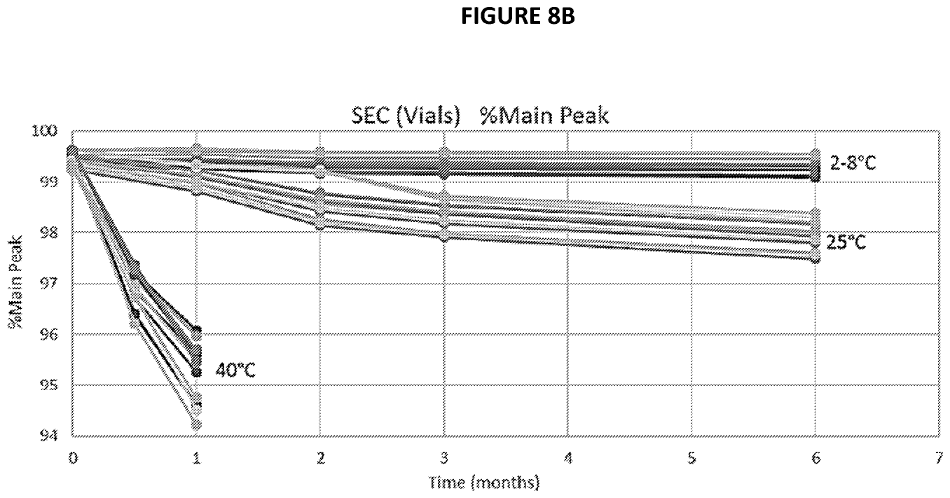

Samples of the stored material were tested via size exclusion chromatography (SEC) to determine the stability of the formulation at various storage time points and at various storage temperatures. The percentage of the main peak of tezepelumab is shown in the indicated figures. Collectively, FIGS. 4, 5A, 5B, 6A, and 6B demonstrate that tezepelumab remains physically stable after storage under a variety of conditions. Taken together, the stability of the tezepelumab formulation is demonstrated at temperatures ranging from −30 to 40° C. for up to 36 months. Even under stressed storage conditions, greater than or equal to 94% of the main peak was present after 1 month of storage (FIG. 5A).

### Example 8

This example demonstrates the effect of storage conditions on tezepelumab formulations comprising acetate, proline and PS80.

To study the effects on stability upon storage at different temperatures and different times of tezepelumab formulations, as well as evaluating the robustness of the formulation, 15 formulations comprising acetate, proline, polysorbate 80, and approximately 110 mg/ml, tezepelumab were made and then underwent simulated transportation testing.

For each formulation, an initial solution comprising tezepelumab at 140 mg/mL was dialyzed against a unique diafiltration (DF) buffer to achieve a complete buffer exchange. Following the buffer exchange, the solution was concentrated to the target tezepelumab concentration and diluted if necessary using a dilution buffer. Unless noted otherwise, the dilution buffer was the same as the DF buffer. After concentration (or concentration and dilution), a surfactant was added.

Target and final (measured) compositions of each formulation (or buffer if noted) used in the study is shown in Table 6 and Table 7, respectively.

TABLE 6
Target pH and Excipient Concentrations for Robustness Study

| Formulation | | Protein | Acetate | L-Proline | Polysorbate 80 |
|---|---|---|---|---|---|
| Number | PH | (mg/mL) | (mM) ^{a} | (% w/v) ^{a} | (% w/v) |
| 1 (static) ^{b} | 5.2 | 110 | 10 | 3.0 | 0.010 |
| 2 | 5.2 | 110 | 10 | 3.0 | 0.010 |
| 3 | 5.2 | 110 | 10 | 3.0 | 0.010 |
| 4 | 4.9 | 99 | 10 | 2.7 | 0.010 |
| 5 | 5.5 | 99 | 10 | 2.7 | 0.010 |
| 6 | 4.9 | 99 | 10 | 3.3 | 0.010 |
| 7 | 5.5 | 99 | 10 | 3.3 | 0.010 |
| 8 | 4.9 | 121 | 10 | 2.7 | 0.010 |
| 9 | 5.5 | 121 | 10 | 2.7 | 0.010 |
| 10 | 4.9 | 121 | 10 | 3.3 | 0.010 |
| 11 | 5.5 | 121 | 10 | 3.3 | 0.010 |
| 12 | 5.2 | 110 | 20 | 3.0 | 0.010 |
| 13 | 5.2 | 110 | 10 | 3.0 | 0.010 |
| 14 | 5.2 | 110 | 10 | 3.0 | 0.005 |
| 15 | 5.2 | 110 | 10 | 3.0 | 0.015 |

^{a} The excipient level listed in the table represents the excipient concentration in the diafiltration dilution buffer.
^{b} Non-transported center point formulation sample

TABLE 7
Measured pH and Excipient Concentrations for Robustness Study

| | | | | | | Polysor- |
|---|---|---|---|---|---|---|
| Formulation | | Protein | Acetate | L-Proline | L-Proline | bate 80 |
| Number | pH | (mg/mL) | (mM) | (% w/v) ^{a} | (mM) ^{a} | (% w/v) |
| 1 (static) ^{b} | 5.2 | 110.3 | 24.5 | 2.4 | 206 | 0.009 |
| 2 | 5.2 | 112.0 | 24.5 | 2.5 | 217 | 0.009 |
| 3 | 5.2 | 110.6 | 24.5 | 2.4 | 205 | 0.008 |
| 4 | 5.0 | 101.3 | 25.6 | 2.2 | 192 | 0.008 |
| 5 | 5.5 | 99.9 | 20.1 | 1.8 | 157 | 0.010 |
| 6 | 5.0 | 100.0 | 27.2 | 2.9 | 254 | 0.008 |
| 7 | 5.5 | 99.2 | 20.6 | 2.7 | 231 | 0.008 |
| 8 | 5.1 | 119.3 | 28.7 | 2.1 | 185 | 0.008 |
| 9 | 5.5 | 119.5 | 22.0 | 2.0 | 175 | 0.009 |
| 10 | 5.1 | 122.3 | 29.2 | 2.8 | 240 | 0.009 |
| 11 | 5.5 | 118.4 | 22.1 | 2.6 | 227 | 0.009 |
| 12 | 5.2 | 115.4 | 31.3 | 1.9 | 161 | 0.009 |
| 13 | 5.2 | 119.7 | 23.4 | 2.3 | 199 | 0.010 |
| 14 | 5.2 | 121.0 | 23.5 | 2.4 | 206 | 0.005 |
| 15 | 5.2 | 120.3 | 23.4 | 2.4 | 207 | 0.015 |

^{a} The L-proline concentrations in % w/v and mM are equivalent.
^{b} Non-transported center point formulation sample

The formulations were tested for stability upon storage by a range of assays used to assess product quality, or a combination thereof. Analysis was carried out on both prefilled syringes (PFS) and vials. Formulations 1 through 15 were compared to analyze the combined effect of protein concentration, pH, acetate, L-proline, and PS80 concentration on protein stability over time. The statistical analysis was performed to assess product quality indicators: SE UHPLC (HMW and main peaks), CEX-UHPLC (acidic, main, and basic peaks), and rCE SDS (HC+LC). Samples were tested via size exclusion chromatography (SEC) and CEX to determine the stability of the formulation at various storage time points. Percentage of high molecular weight (HMW) species and percentage of the main peak for each formulation was reported. The main peak percentage reflected the amount of tezepelumab (in monomer form) that remained after the indicated storage period. Capillary electrophoresis-sodium dodecyl sulfate (CE-SDS) is used for separation of denatured protein size variants under non-reduced (nrCE-SDS) or reduced conditions (rCE-SDS). Under rCE-SDS, the sum of % heavy chain and light chain were measured.

The analysis was performed based on the change in peak percentage from time zero for each of the time points. For stability, samples of each formulation were filled into containers and then stored for up to 6 months (e.g., 1 week, 2 weeks, 4 weeks, 3 months, 6 months) at a temperature of about 2-8° C. to about 25° C., or 1 month at a temperature of about 40° C.

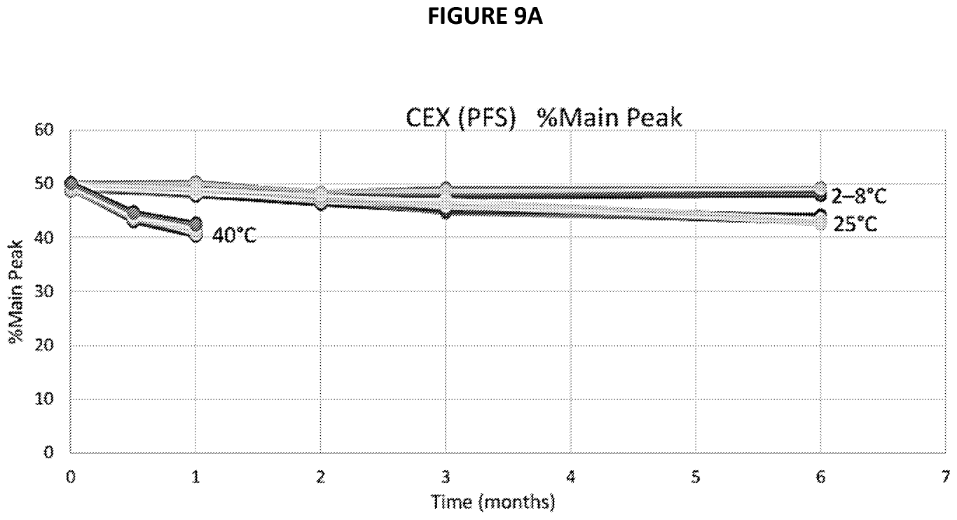

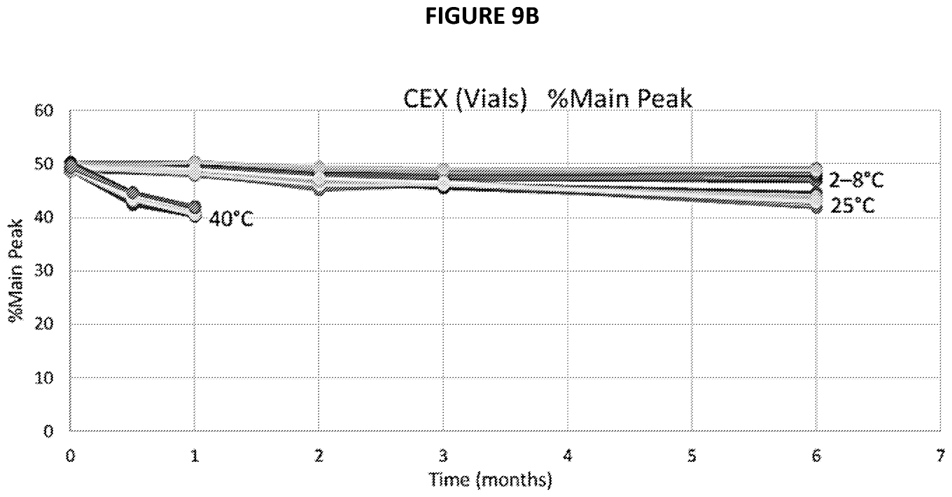

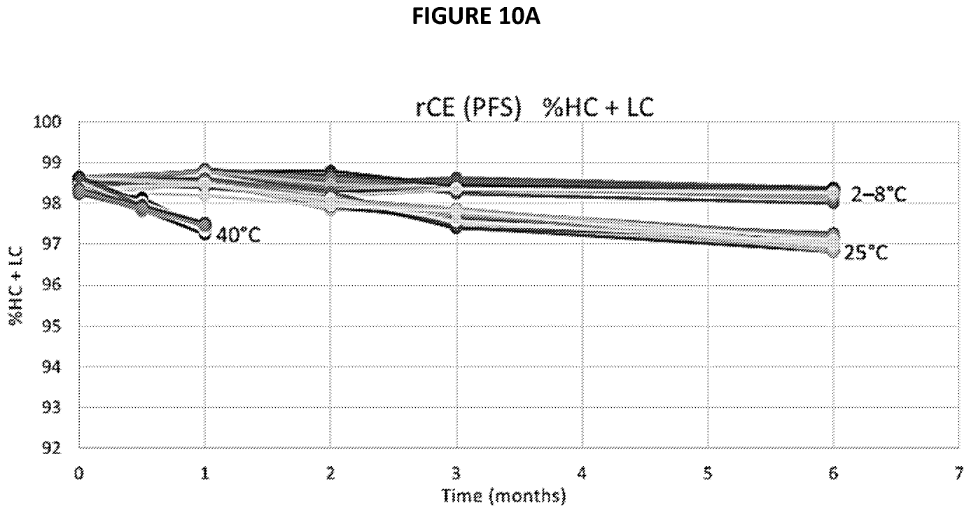

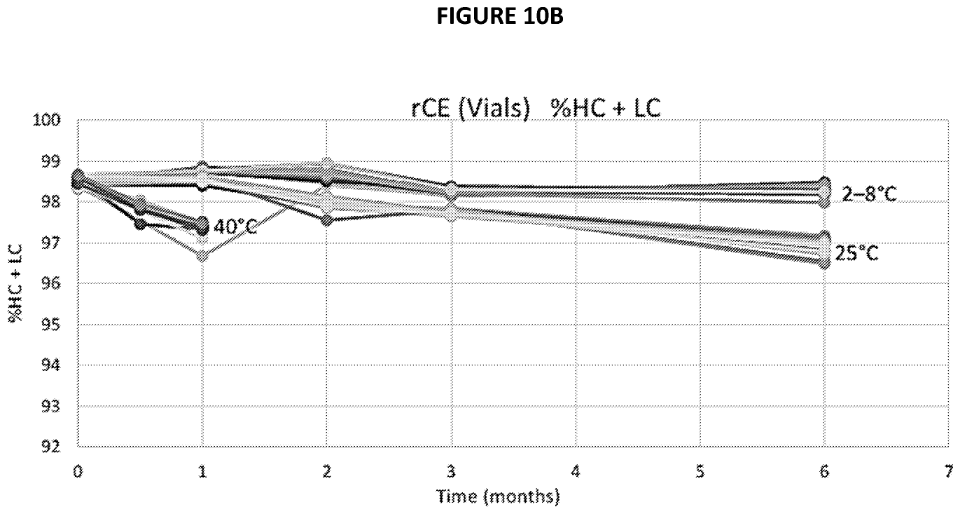

SEC and CEX results are shown in FIGS. 7 to 9. SEC results are reported as percentages (%) for main peak (monomer) (FIG. 7A, 7B) and high molecular weight (HMW) species (FIG. 8A, 8B). CEX results are reported as percentages (%) for main peak (FIG. 9A, 9B). rCE-SDS results are reported as % for heavy chain +light chain (HC +LC) (FIG. 10A, 10B). Samples stored at 2° C. to 8° C. for 6 months showed only minor changes.

Particle formation was also measured by HIAC (High Accuracy liquid particle counting) and micro-flow imaging (MFI). No subvisible particle trends were observed at 2° C. to 8° C. and the majority of particles were determined to be related to the primary container used for storing the material. No visible proteinaceous particles were observed in the formulations.

While the study noted some statistically significant dependence of degradation on pH and acetate concentration for samples stored for 6 months at 25° C., the overall changes observed were minor in comparison to the rate of degradation due to time. The drug product formulation is robust to minor variations in formulation composition when pH, protein concentration, amount of polysorbate 80, acetate and proline each varies around the center point. The 15 formulations were all physically and chemically stable when stored at the recommended storage temperature of 2-8° C. for 6 months, as expected. Variations in parameters such as protein concentration, acetate and proline concentration, pH, and polysorbate 80 concentrations and storage container did not show significant impact.

Numerous modifications and variations of the invention as set forth in the above illustrative examples are expected to occur to those skilled in the art. Consequently only such limitations as appear in the appended claims should be placed on the invention.

What is claimed is:

1. A composition comprising greater than about 100 mg/mL of an anti-TSLP antibody, a surfactant, proline, and a buffer,
wherein the anti-TSLP antibody comprises
(A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and
(B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

2. The composition of claim 1, wherein the anti-TSLP antibody comprises:
(A) a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11; or
iii. a sequence of amino acids encoded by a polynucleotide that hybridizes under moderately stringent conditions to the complement of a polynucleotide consisting of SEQ ID NO:11; or
(B) a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9; or
iii. a sequence of amino acids encoded by a polynucleotide that hybridizes under moderately stringent conditions to the complement of a polynucleotide consisting of SEQ ID NO:9; or
(C) a light chain variable domain of (A) and a heavy chain variable domain of (B).

3. The composition of claim 1 or 2, wherein the anti-TSLP antibody is an IgG2 antibody.

4. The composition of any one of claims 1 to 3, wherein the anti-TSLP antibody comprises a heavy chain comprising the amino acid sequence of SEQ ID NO: 13, a light chain comprising the amino acid sequence of SEQ ID NO: 14, or a heavy chain comprising the amino acid sequence of SEQ ID NO: 13 and a light chain comprising the amino acid sequence of SEQ ID NO: 14.

5. The composition of any one of claims 1 to 4, wherein the anti-TSLP antibody specifically binds to a TSLP polypeptide as set forth in amino acids 29-159 of SEQ ID NO: 2.

6. The composition of any one of claims 1 to 5, wherein both binding sites of the anti-TSLP antibody have identical binding to TSLP.

7. The composition of any one of claims 1 to 6, wherein the anti-TSLP antibody is present in the composition at a concentration less than about 200 mg/mL.

8. The composition of claim 7, wherein the anti-TSLP antibody is present in the composition at a concentration less than about 150 mg/mL.

9. The composition of claim 8, wherein the anti-TSLP antibody is present in the composition at a concentration of about 110 mg/mL to about 140 mg/mL.

10. The composition of claim 9, wherein the anti-TSLP antibody is present in the composition at a concentration about 110 mg/mL ±10%.

11. The composition of claim 9, wherein the anti-TSLP antibody is present in the composition at a concentration about 140 mg/mL ±10%.

12. The composition of any one of the preceding claims, wherein the surfactant is amphipathic and nonionic.

13. The composition of claim 12, wherein the surfactant is a polysorbate.

14. The composition of claim 13, wherein the surfactant is polysorbate 20 or polysorbate 80 or a mixture thereof.

15. The composition of any one of claims 12 to 14, comprising a surfactant at a concentration less than or about 0.005% (w/v) to about 0.015% (w/v).

16. The composition of claim 15, comprising about 0.010% (w/v) ±0.0025% (w/v) surfactant.

17. The composition of claim 15, comprising about 0.005% (w/v), 0.010% (w/v), or 0.015% (w/v) surfactant.

18. The composition of any one of the preceding claims, comprising less than about 3.0% (w/v) proline.

19. The composition of any one of the preceding claims, comprising about 2.4% (w/v) to about 2.8% (w/v) proline or about 2.5% (w/v) to about 2.8% (w/v) proline.

20. The composition of any one of the preceding claims, wherein the proline is L-proline.

21. The composition of any one of the preceding claims, wherein proline is the only amino acid present in the composition.

22. The composition of any one of the preceding claims, wherein the buffer is selected from the group consisting of: succinate, glutamate, histidine, and acetate.

23. The composition of claim 22, wherein the buffer is acetate.

24. The composition of any one of the preceding claims, comprising about 10 mM to about 30 mM buffer.

25. The composition of claim 24, comprising about 20 mM to about 28 mM buffer, optionally, about 23 mM to about 28 mM or about 24 mM to about 28 mM.

26. The composition of claim 25, comprising about 24 mM to about 26 mM buffer.

27. The composition of any one of the preceding claims, comprising not more than 0.001% (w/v) of a sugar or citrate, optionally, wherein the sugar is a disaccharide, e.g., trehalose and sucrose.

28. The composition of any one of the preceding claims, wherein the composition is a liquid.

29. The composition of claim 28, wherein the pH is less than about 6.0, optionally, less than about 5.5.

30. The composition of claim 29, wherein the pH is about 4.5 to about 5.5.

31. The composition of claim 30, wherein the pH is about 4.8 to about 5.4.

32. The composition of claim 31, wherein the pH is about 4.9, 5.2, or 5.4.

33. The composition of any one of claims 28 to 32, characterized by a reduced viscosity, relative to liquid composition not comprising proline.

34. The composition of any one of claims 28 to 33, characterized by a viscosity of less than about 24 cP at about 20° C. to about 25° C. when the concentration of the anti-TSLP antibody is less than 155 mg/mL, optionally, ˜6 cP when the concentration of the anti-TSLP antibody is about 110 mg/mL or about 15 cP when the concentration of the anti-TSLP antibody is about 140 mg/mL.

35. The composition of claim 29, characterized by a viscosity of about 5 cP to about 20 cP, optionally about 15 cP.

36. The composition of any one of claims 28 to 35, wherein the composition is isotonic or has an osmolality in a range of about 200 mOsm/kg to about 500 mOsm/kg, or about 225 mOsm/kg to about 400 mOsm/kg, or about 250 mOsm/kg to about 350 mOsm/kg.

37. The composition of any one of the previous claims, wherein less than 5% of the antibody is degraded after about 24 months to about 36 months of storage at 2° C. to 8° C. as determined by Size Exclusion Chromatography (SEC), optionally, wherein less than 2% of the antibody is degraded after 24 months or 36 months of storage at 2° C. to 8° C.

38. The composition of any one of the previous claims, wherein less than 5% of the antibody is degraded after at least 2 weeks (optionally, after at least 1 month, after at least 2 months, after at least 3 months, after at least 4 months, after at least 5 months or after at least 6 months) of storage at about 25° C., as determined by SEC.

39. The composition of any one of the previous claims, wherein less than 5% of the antibody is degraded after about 24 months to about 36 months of storage at 2° C. to 8° C. followed by at least 2 weeks or at least 1 month or at least 2 months of storage at about 25° C., as determined by SEC.

40. A composition comprising, about 110 mg/mL to about 140 mg/mL tezepelumab, about 0.01% (w/v) ±0.005% (w/v) polysorbate 80, about 2.4% (w/v) to about 2.8% (w/v) L-proline, and about 20 mM to about 28 mM acetate, wherein the viscosity of the composition is less than about 20 cP and the pH is less than about 5.5.

41. The composition of claim 40, wherein the pH is 5.2, optionally, wherein the viscosity is about 15 cP at 20° C. to about 25° C.

42. A composition comprising about 110 mg/mL of an anti-TSLP antibody, 0.01% (w/v) polysorbate 80, about 2.4% (w/v) to about 2.8% (w/v) L-proline, and about 20 mM to about 28 mM acetate, wherein the composition has a pH of about 5.2.

43. The composition of claim 42, comprising about 22 mM to about 26 mM.

44. The composition of claim 43, comprising about 24 mM to about 26 mM acetate.

45. The composition of any one of claims 42 to 44, comprising 110 mg/mL anti-TSLP antibody, 24 mM acetate, 2.5% (w/v) L proline, and 0.01% (w/v) polysorbate 80 at pH 5.2.

46. The composition of claim 1, comprising 110 mg/mL anti-TSLP antibody, 10 mM acetate, 3.0% (w/v) L-proline, and 0.01% (w/v) polysorbate 80, at pH 5.2.

47. A composition comprising about 140 mg/mL of an anti-TSLP antibody, 0.01% (w/v) polysorbate 80, about 2.5% (w/v) to about 2.8% (w/v) L-proline, and about 20 mM to about 28 mM acetate, wherein the composition has a pH of about 5.2.

48. The composition of claim 47, comprising about 25 mM to about 26 mM acetate.

49. An article of manufacture comprising the composition of any one of the preceding claims optionally, comprising about 0.5 mL to about 5 mL of the composition.

50. The article of manufacture of claim 49, comprising about 1 mL to about 3 mL of the composition.

51. A prefilled syringe comprising the composition of any one of the preceding claims optionally, comprising about 0.5 mL to about 5 mL of the composition.

52. The prefilled syringe of claim 51, comprising about 1 mL to about 3 mL of the composition.

53. A vial comprising the composition of any one of the preceding claims, optionally, comprising about 0.5 mL to about 5 mL of the composition.

54. The vial of claim 53, comprising about 1 mL to about 3 mL of the composition.

55. An autoinjector comprising the composition of any one of the preceding claims, optionally, comprising about 0.5 mL to about 5 mL.

56. The autoinjector of claim 55, comprising about 1 mL to about 3 mL of the composition.

57. The autoinjector of claim 55 or 56, wherein the auto-injector is an Ypsomed YpsoMate®.

58. The autoinjector of any one of claims 55 to 57, wherein the auto-injector is disclosed in WO 2018/226565, WO 2019/094138, WO 2019/178151, WO 20120/072577, WO2020/081479, WO 2020/081480, PCT/US20/70590, PCT/US20/70591, PCT/US20/53180, PCT/US20/53179, PCT/US20/53178, or PCT/US20/53176.

59. A method for treating an inflammatory disease in a subject comprising administering to the subject a therapeutically effective amount of the composition of any one of the preceding claims.

60. The method of claim 59, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

61. The method of claim 60 wherein the inflammatory disease is asthma.

62. The method of claim 59 or 60, comprising administering the composition at an interval of every 2 weeks.

63. The method of claim 59 or 60, comprising administering the composition every 4 weeks.

64. The method of any one of claims 59 to 63, wherein the composition is administered for a period of at least 4 months, 6 months, 9 months, 1 year or more.

65. The method of any one of claims 59 to 64, wherein the composition is administered subcutaneously.

66. The method of any one of claims 61 to 65, wherein the asthma is severe asthma.

67. The method of any one of claims 61 to 66, wherein the asthma is eosinophilic or non-eosinophilic asthma.

68. The method any one of claims 61 to 67, wherein the asthma is low eosinophil asthma.

69. The method of any one of claims 59 to 68, wherein the subject is an adult.

70. The method any one of claims 59 to 68, wherein the subject is a child or adolescent.

71. The method any one of claims 59 to 70, wherein the administration decreases eosinophils in blood, sputum, broncheoalveolar fluid, or lungs of the subject.

72. The method any one of claims 59 to 71, wherein the administration shifts cell counts in the subject from a Th2 high population to a Th2 low population.

73. The method any one of claims 59 to 72, wherein the administration improves one or more measures of asthma in a subject selected from the group consisting of forced expiratory volume (FEV), FEV1 reversibility, forced vital capacity (FCV), FeNO, Asthma Control Questionnaire-6 score and AQLQ(S)+12 score.

74. The method any one of claims 59 to 73, wherein the administration improves one or more symptoms of asthma as measured by an asthma symptom diary.

75. A method of making a stable, liquid antibody composition having a viscosity of less than about 24 cP and comprising less than about 200 mg/mL an anti-TSLP antibody, a surfactant and a buffer, said method comprising: (i) combining a first solution comprising the antibody at a first concentration, acetate and proline with a buffer comprising acetate and proline, to obtain a solution comprising about 110 mg/mL to about 140 mg/mL tezepelumab, proline, and acetate, and (ii) adding a surfactant to achieve a final concentration of about 0.01% (w/v)±0.005% (w/v) surfactant.

76. The method of claim 75, wherein the stable, liquid composition comprises about 140 mg/mL of an anti-TSLP antibody.

77. The method of claim 75 or 76, wherein the viscosity of the stable, liquid composition with the proline is reduced relative to the stable, liquid composition without the proline.

78. The method of any one of claims 75 to 77, wherein the viscosity of the stable, liquid composition is less than about 20 cP.

79. The method of any one of claims 75 to 78, wherein a solution comprising about 200 mM to about 300 mM proline is combined with the first solution.

80. The method of any one of claims 75 to 79, wherein the proline is L-proline.

81. The method of any one of claims 75 to 80, wherein the surfactant is polysorbate 80 or polysorbate 20.

82. The method of claim 81, wherein the surfactant is polysorbate 80.

83. The method of any one of claims 75 to 82, wherein the buffer is made with glacial acetic acid.

84. The method of any one of claims 75 to 83, wherein the buffer composition comprises about 1 mM to about 30 mM acetate, optionally, about 5 mM to about 15 mM acetate.

85. The method of any one of claims 75 to 84, wherein the pH of the stable, liquid antibody composition is about 5.2.

86. The method of any one of claims 75 to 85, wherein the anti-TSLP antibody comprises:
(A) a light chain variable domain selected from the group consisting of:
iv. a sequence of amino acids at least 80% identical to SEQ ID NO:12;
v. a sequence of amino acids encoded by a polynucleotide sequence that is at least 80% identical to SEQ ID NO:11; or
vi. a sequence of amino acids encoded by a polynucleotide that hybridizes under moderately stringent conditions to the complement of a polynucleotide consisting of SEQ ID NO:11; or
(B) a heavy chain variable domain selected from the group consisting of:
iv. a sequence of amino acids that is at least 80% identical to SEQ ID NO:10;
v. a sequence of amino acids encoded by a polynucleotide sequence that is at least 80% identical to SEQ ID NO:9; or
vi. a sequence of amino acids encoded by a polynucleotide that hybridizes under moderately stringent conditions to the complement of a polynucleotide consisting of SEQ ID NO:9; or
(C) a light chain variable domain of (A) and a heavy chain variable domain of (B).

87. The method of claim 86, wherein the anti-TSLP antibody is an IgG2 antibody.

88. The method of claim 86 or 87, wherein the anti-TSLP antibody comprises a heavy chain comprising the amino acid sequence of SEQ ID NO: 13, a light chain comprising the amino acid sequence of SEQ ID NO: 14, or a heavy chain comprising the amino acid sequence of SEQ ID NO: 13 and a light chain comprising the amino acid sequence of SEQ ID NO: 14.

89. A method of making a stable, liquid antibody composition having a viscosity of less than about 24 cP and comprising less than about 200 mg/mL an anti-TSLP antibody, a surfactant and a buffer, said method comprising formulating the anti-TSLP antibody with a buffer comprising about 10 mM to about 20 mM acetate and about 2.7% (w/v) to about 3.3% (w/v) having a pH of about 4.9 to about 5.5, and (ii) adding a surfactant to achieve a final concentration of about 0.005% (w/v) ±0.015% (w/v) surfactant.

90. The method of claim 89, wherein the buffer is made using glacial acetic acid.

91. The method of claim 89 or 90, wherein the buffer is titrated to pH 5.2 using sodium hydroxide.

92. A solution for injection (i) comprising about 110 mg/mL to about 115 mg/mL tezepelumab, about 24 mM to about 26 mM acetate made using glacial acetic acid, about 2.4% to about 2.6% (w/v) L-proline, about 0.01% polysorbate 80, sodium hydroxide, and water for injection, (ii) having a pH of about 5.2 and a shelf-life of about 3 years.

93. A prefilled syringe comprising about 1.91 mL of the stable, liquid antibody composition of claim 92.
